(19) United States
(12) Patent Application Publication
(10) Pub. No.: US 2016/0011290 A1
(43) Pub. Date: Jan. 14, 2016

(54) Non-Invasive, In-Vivo Measurement of Blood Constituents Using a Portable Nuclear Magnetic Resonance Device

(71) Applicant: Victor IANNELLO, (US)

(72) Inventor: Victor Iannello, Roanoke, VA (US)

(21) Appl. No.: 14/771,535

(22) PCT Filed: May 21, 2014
(86) PCT No.: PCT/US14/38998

§ 371 Date: Aug. 31, 2015

Related U.S. Application Data
(60) Provisional application No. 61/825,689, filed on May 21, 2013.

(51) Int. Cl. G01R 33/561; A61B 5/145; G01R 33/48; G01R 33/44; G01R 33/36; G01R 33/565; A61B 5/055; A61B 5/00

(52) U.S. Cl. CPC G01R 33/5615; A61B 5/055; A61B 5/14532; A61B 5/14546; A61B 5/4875; A61B 5/6826; G01R 33/448; G01R 33/36; G01R 33/56527; G01R 33/4828; G01R 33/56563

(57) ABSTRACT

Certain exemplary embodiments can provide a system, machine, device, manufacture, circuit, composition of matter, and/or user interface adapted for and/or resulting from, and/or a method and/or machine-readable medium comprising machine-implementable instructions for, activities that can comprise and/or relate to, applying a static magnetic field induced by one or more permanent magnets to a cup that is configured to receive at least a portion of a digit of an animal.

### CROSS-REFERENCES TO RELATED APPLICATIONS

This application claims priority to, and incorporates by reference herein in its entirety, pending U.S. Provisional Patent Application 61/825,689 (Attorney Docket 1176-003), filed 21 May 2013.

### BRIEF DESCRIPTION OF THE DRAWINGS

A wide variety of potential, feasible, and/or useful embodiments will be more readily understood through the herein-provided, non-limiting, non-exhaustive description of certain exemplary embodiments, with reference to the accompanying exemplary drawings in which:

FIG. 1 is an exemplary graph of the ¹H spectrum of human blood;

FIG. 2 is an exemplary graph of the relationship between T₁ and glucose concentration in human blood;

FIG. 3 is an exemplary graph of a CPMG pulse sequence;

FIG. 4 is an exemplary graph of multiple echo trains;

FIG. 5 shows exemplary T₂ distribution curves for oil-water mixture of various concentrations;

FIG. 6 is a longitudinal cross-section, taken at section A-A of FIG. 7, of an exemplary embodiment of a non-invasive, in-vivo instrument for measuring the constituents of blood;

FIG. 7 is a side cross-section, taken at section B-B of FIG. 6, of an exemplary embodiment of a NMR instrument;

FIG. 8 is a cross-section of a tip of an exemplary human finger, taken along a longitudinal axis of the finger;

FIG. 9 is an exemplary graph of resonant absorption in an exemplary slice of an exemplary human finger;

FIG. 10 is a flowchart of an exemplary embodiment of a method;

FIG. 11 is a flowchart of an exemplary embodiment of a method;

FIG. 12 is a flowchart of an exemplary embodiment of a method;

FIG. 13 is a block diagram of an exemplary embodiment of a system, device, and/or instrument;

FIG. 14 is a plot of exemplary data;

FIG. 15 is a block diagram of an exemplary embodiment of a system;

FIG. 16 is a block diagram of an exemplary embodiment of an information device; and

FIG. 17 is a flowchart of an exemplary embodiment of a method.

### DESCRIPTION

Certain exemplary embodiments can relate to a device and/or method for non-invasively measuring the constituents of human blood in vivo. Certain exemplary embodiments can be used to monitor the concentration of critical components such as the level of glucose, cholesterol, and/or alcohol. Certain exemplary embodiments can be relatively small and/or inexpensive, and/or can be suitable for use at home and/or in a small medical office. Certain exemplary embodiments can use the principles of nuclear magnetic resonance (NMR) to measure blood components.

The measurement of blood glucose levels can be important in the detection and/or management of diabetes. The incidence of diabetes is dramatically increasing in the United States and throughout the world. For instance, the Center for Disease Control and Prevention (CDC) estimates that nearly 26 million Americans have diabetes, and an additional 79 million U.S. adults have pre-diabetes (CDC, 2011). Pre-diabetes raises a person's risk of Type 2 diabetes, heart disease and stroke. The CDC projects that as many as one in three U.S. Adults could have diabetes by 2050 if the current trend continues. Type 2 diabetes, in which the body gradually loses its ability to produce insulin, accounts for 90 to 95 percent of diabetes cases. Contributing factors include age, obesity, genetics, having diabetes while pregnant, and sedentary lifestyle.

Today, the most common methods for measuring glucose levels require blood samples taken from the body. The blood is introduced to a test strip with a reducing enzyme such as glucose oxidase or hexokinase, and the reaction to the blood glucose is quantified. However, the collection of a blood sample can be painful and/or inconvenient, especially if required multiple times per day. Also, the test strips are consumables that add to the expense of measurement.

It can be desirable to have a portable instrument that can safely and/or accurately measure the constituents of blood in a non-invasive manner. By reducing the size and/or cost of the instrument, it can be suitable for use at home and/or in a medical office. Certain exemplary embodiments described herein were conceived with this in mind.

### Principles of NMR

The nuclei of isotopes with an odd number of neutrons and protons exhibit a net magnetic moment and angular momentum or spin. Some isotopes that exhibit magnetic moments include hydrogen (¹H), carbon (¹³C), and sodium (²³Na). The ¹H nucleus, which is a single proton, can have a particular significance. It is abundant in water and organic compounds, and has a relatively large magnetic moment. Certain exemplary embodiments rely on the magnetic resonance of this isotope.

The NMR effect can occur by first applying a steady magnetic field Bₒ to a sample. Because of the magnetic moment of the hydrogen nuclei, the spin axes will tend to align with the applied magnetic field, and precess about Bₒ at a frequency fₒ known as the Larmor frequency. This frequency can be calculated with the following equation:

fₒ=γBₒ/2π (1)

In the equation, γ is the gyromagnetic ratio, which is a measure of the magnetic moment. For a hydrogen nucleus, γ/2π is 42.58 MHz/T, which means that if the applied field is 1 T, the Larmor frequency is 42.58 MHz. Because the Larmor frequency depends on the applied field, any technique that measures the Larmor frequency in order to discriminate between different chemical species typically very precisely controls this applied field.

Typically, the second step in using NMR is to cause the hydrogen nuclei to tip away from the alignment of the applied field, Bₒ. This tipping can occur by applying a time-varying field B₁ perpendicular to Bₒ. If a time varying field is applied as a pulse with a duration τₚ and the frequency of the field B₁ matches the Larmor frequency, then the nuclei will tip towards the transverse with an angle given by

θ=γ B₁ τₚ (2)

By varying the strength and duration of the radio frequency (RF) pulse, the tip angle can be controlled. Commonly, the strength of a pulse is described by the tip angle, e.g., a 90-degree pulse, or a 180-degree pulse. Therefore, a 90-degree pulse would tip the spin axis to a transverse plane while a 180-degree pulse would tip the spin axis in a direction anti-parallel to the applied field Bₒ.

After the nuclei are tipped, they continue to precess at the Larmor frequency. This precession causes time-varying magnetic fields, which can be detected. However, the precessing nuclei lose coherence with time, and therefore the magnetic field also decays. This decay in coherence is known as relaxation.

This decay is comprised of two components. Subsequent to the RF pulse, the nuclei tend to realign with the applied field Bₒ with a time constant T₁, also known as the spin-lattice time constant. This constant generally governs how quickly the sample returns to the initial equilibrium state, and can vary for different molecules. Similarly, the component of the magnetic moment in the transverse plane can lose its coherence with a relaxation time constant referred to as the free induction decay (FID) time constant T₂*. This time constant is likely due to inhomogeneity of the magnetic field and/or to certain molecular processes. If the effects of field inhomogeneity are eliminated, the relaxation time constant typically increases to a value known as the transverse (spin-spin) relaxation time constant T₂, which is generally a characteristic of the molecule and independent of gradients in the applied field.

An NMR instrument can operate by exposing the sample to a static Bₒ field and then pulsing the sample one or more times with RF signals to tip the magnetic moments of nuclei. After pulsing, the nuclei begin to relax, and these decaying oscillations are detected by a sensor antenna. The signal from these oscillations is acquired and numerically processed. The acquired signal represents a superposition of signals of various amplitudes, relaxations, and/or frequencies.

Although it is stated above that the Larmor frequency for a hydrogen nucleus is a function only of the applied field Bₒ, in fact, the Larmor frequency is more accurately a function of the local field that the nucleus experiences. This local field can slightly differ from the applied field due to the shielding effects of electrons and/or coupling effects between hydrogen nuclei in a molecule. This shift in Larmor frequency is known as chemical shift, and can allow the signatures of different molecules to be conventionally detected using frequency spectrum techniques. The chemical shifts can be less than approximately 20 ppm (i.e., 20 Hz/MHz), and typically can be less than approximately 8 ppm. The small shifts can require extremely uniform fields in order to resolve the individual resonance peaks due to the chemical species.

### Measurement of Glucose Levels Using NMR Chemical Shift

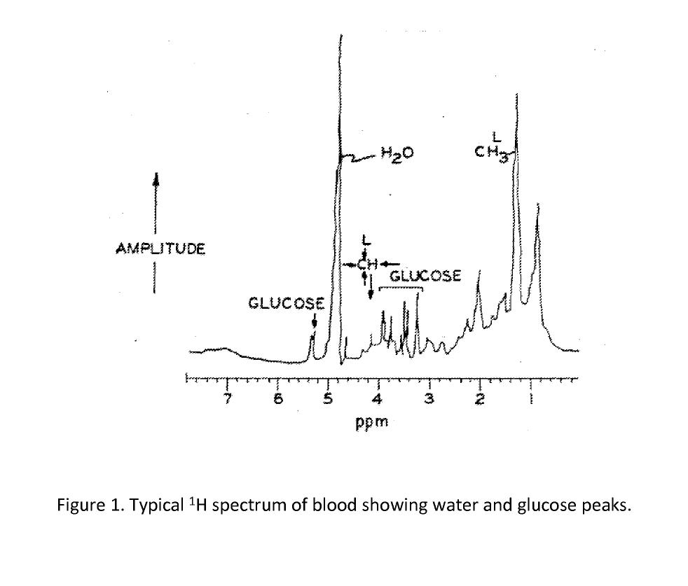

Certain exemplary embodiments can include measuring glucose levels in blood using NMR spectroscopy. The ¹H spectrum of blood is shown in FIG. 1. Resonances due to chemical shifts of water (e.g., 4.79 ppm), glucose (e.g., 5.25 ppm), and lactate (e.g., 1.34 ppm) are clearly visible. To determine blood glucose levels, a device that provides a configuration of permanent magnets can be used to create the steady Bₒ field with only a very small amount of magnetic field leaking outside of the device. After a finger is inserted into the device, and an NMR spectrum can be obtained. The glucose level in the blood then can be determined by calculating the area under the glucose peak relative to the area under the water peak. This ratio then can be compared against a standard to obtain the actual glucose level. Yet certain exemplary embodiments can require a high level of uniformity of the magnetic field (less than 0.2 ppm) to resolve the chemical shifts with a reasonable degree of accuracy.

### Measurement of Glucose Levels Using Spin-Lattice Relaxation

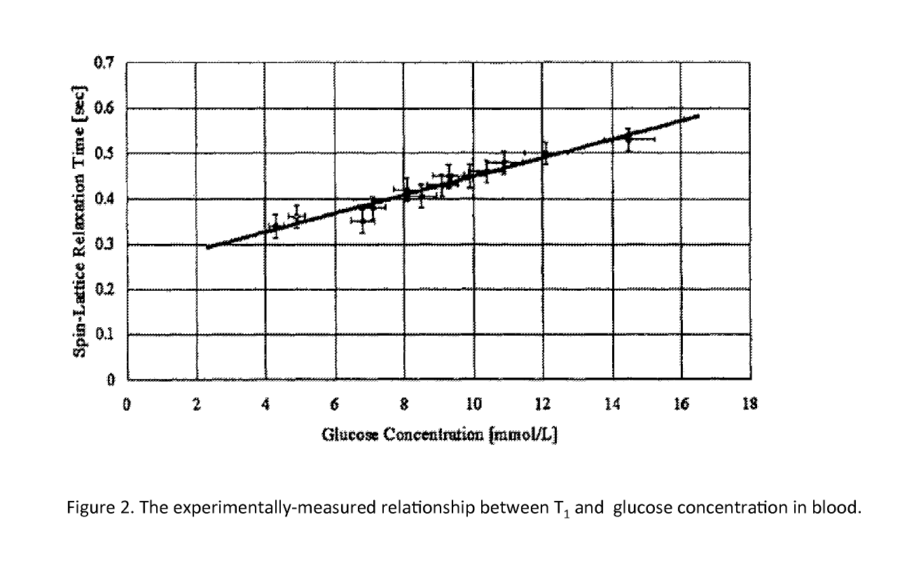

Certain exemplary embodiments can utilize a small NMR for measuring glucose levels in blood by measuring the spin-lattice relaxation time (T₁). The relationship between T₁ and glucose concentration in blood has been experimentally measured, and is shown in FIG. 2. A configuration of permanent magnets can be used to create a steady, homogeneous Bₒ field. By superimposing a slowing varying field on top of Bₒ, RF pulses are absorbed only when the RF frequency matches the Larmor frequency corresponding to the net B field. Therefore, resonant absorption only occurs at discrete times. By detecting the timing and amplitude of these resonant absorptions, T₁ can be measured. Yet again, certain exemplary embodiments can require a high degree of field uniformity to obtain the signal-to-noise ratio that is desired to accurately measure T₁ in this manner. Also, because other blood constituents other than glucose have an effect on T₁, the accuracy of the glucose measurement can be strongly affected by variations in other blood constituents.

### NMR in Non-Homogeneous Fields Using Spin-Echo Detection

Certain exemplary embodiments can provide a precise, homogeneous Bₒ field in order to obtain the resolution desired for measurements of spectral peaks due to chemical shifts or for the measurement of spin-lattice relaxation. If the chemical species of blood can be identified using NMR techniques that are suitable for inhomogeneous fields, then a drastic reduction in complexity, size, and/or cost of the NMR measurement device can result.

Inhomogeneity in the Bₒ field can cause rapid decay of the oscillating magnetic moment in the transverse plane because hydrogen nuclei at difference locations can see a different local B-field, and will therefore precess with different Larmor frequencies. In time, such frequency difference leads to phase difference, which causes loss of coherence among the hydrogen nuclei, and the signals decay. As a result, the FID time constant T₂* can be as short as tens of microseconds, and it can become difficult to measure and/or to use T₂ as a way to distinguish between chemical species in the sample.

It can be possible to reverse the loss of coherence caused by the inhomogeneous static field.

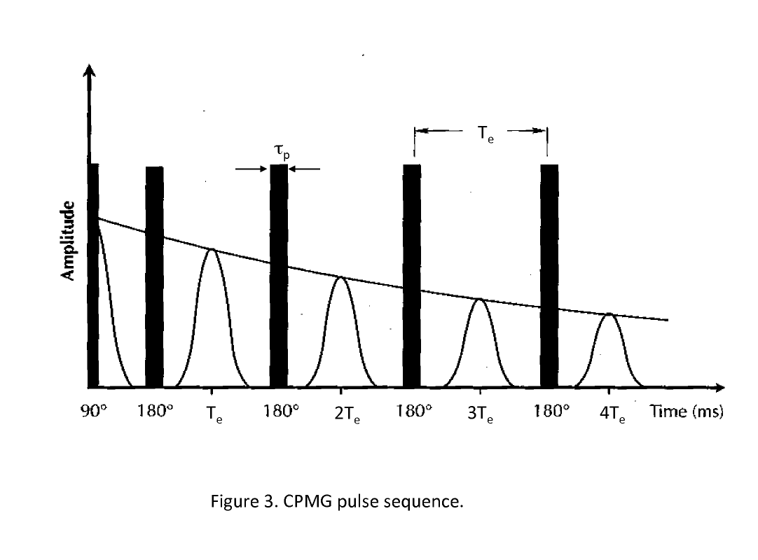

For example, a series of pulses known as a Carr-Purcell-Meiboom-Gill (“CPMG”) pulse sequence, which is shown in FIG. 3, can be applied. To generate what is known as a spin-echo train, a 90-degree pulse first can be applied, followed by 180-degree pulse applied with a delay time τ. After this 180-degree pulse, additional 180-degree pulses are applied with an interval 2τ, also known as the echo time Tₑ. After each 180-degree pulse is applied, the direction of precession of the hydrogen nuclei is reversed such that the phase spread of the nuclei begins to reverse, reaching coherence (focus) at a time τ after the pulse. At this time of coherence, data can be acquired. Extending this concept, 180-degree pulses can be applied at times τ, 3τ, 5τ, etc., and data can be acquired at times 2τ, 4τ, 6τ, etc. The refocusing that occurs at these times can cause what is referred to as an “echo”. By acquiring the echo signals, the effect of inhomogeneity of the Bₒ field can be minimized. As a result, the magnetic moments in the transverse plane attenuate with a time constant T₂ instead of the much smaller T₂*. The value of T₂ then can be determined based on the decay observed with each subsequent acquisition.

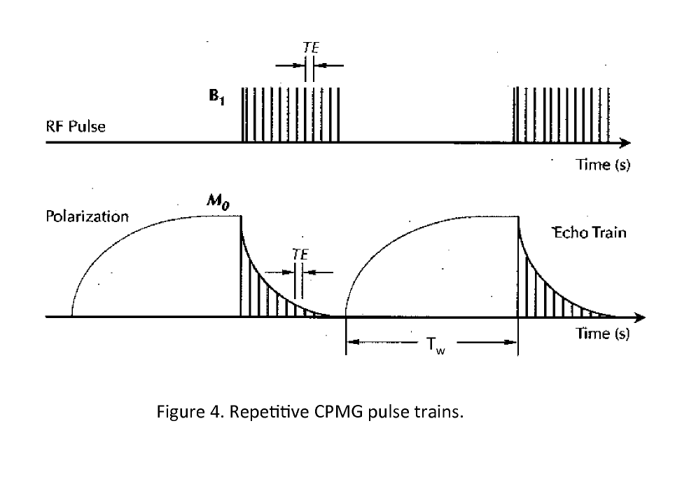

FIG. 4 shows multiple echo trains that can be acquired. After a period equal to several times T₂, the magnetization has reached a small value, and pulse/acquisition sequence is stopped. Once the pulsing has stopped, the magnetization asymptotically builds again to its equilibrium value Mₒ with a time constant T₁. After a wait time T_{w} equal to several times T₁, magnetization is nearly complete, and a new CPMG pulsing sequence can be applied. By repeating this sequence a number of times and averaging the results, the signal-to-noise ratio (SNR) can be greatly improved at the expense of the longer time required to acquire the additional data.

For a molecule with spin-lattice time constant T₁, transverse relaxation time constant T₂, an equilibrium magnetization Mₒ, and a CPMG sequence with echo time Tₑ and wait time T_{w}, the magnetic moment of the echo signal as a function of time can be modeled as:

$$M ( t ) = {M}_{o} ( 1 - {e}^{- \frac{{T}_{w}}{{T}_{1}}} ) ( {e}^{- \frac{t}{{T}_{2}}} ) \qquad ( 3 )$$

Because echo comes into focus only at discrete times tₙ=nTₑ, the peak magnitude of each echo in the train can be modeled as:

$${M}_{n} = {M}_{o} ( 1 - {e}^{- \frac{{T}_{w}}{{T}_{1}}} ) ( {e}^{- \frac{{nT}_{e}}{{T}_{2}}} ) \qquad ( 4 )$$

### Spin-Echo Detection for Multi-Component Systems

In order to distinguish the components of blood, we recognize that the magnetization signal of each constituent can decay with a different value of T₁ and T₂. Therefore, if there are I total components, the magnitude of the echo signals at any time nTₑ represent the sum of the signals from each component i as follows:

$${M}_{n} = {\sum }_{i = 1}^{I} {M}_{o , i} ( 1 - {e}^{- \frac{{T}_{w}}{{T}_{1 , i}}} ) ( {e}^{- \frac{n {T}_{e}}{{T}_{2 , i}}} ) \qquad ( 5 )$$

If the wait time T_{W} is chosen to be several times T₁ of the slowest component of interest, then Eq. (5) simplifies to

$${M}_{n} = {\sum }_{i = 1}^{I} {M}_{o , i} ( {e}^{- \frac{{nT}_{e}}{{T}_{2 , i}}} ) \qquad ( 6 )$$

where each component i is characterized by an initial magnetization M_{o,i} (related to its concentration) and its transverse relaxation constant T_{2,i}. What can be desired is to find the distribution Mₒ versus T₂ at a number of discrete points i, which can be an indication of the relative concentrations of the components of the sample. Thus, we can determine the ordered pairs (M₀, T₂)ᵢ by first assuming distribution of values of T_{2,i} in the sample, e.g., a geometric progression such as 1, 2, 4, 8, . . . , 8192 ms. Then, the unknowns in Eq. 6 can be the initial magnetization of the components M_{o,i} and/or the number of equations can be the number of echoes acquired N (potentially after adding S echoes to improve the SNR). This approach can provide a system of n equations in I unknowns, which in general can be directly and/or non-iteratively solved if N is greater than I.

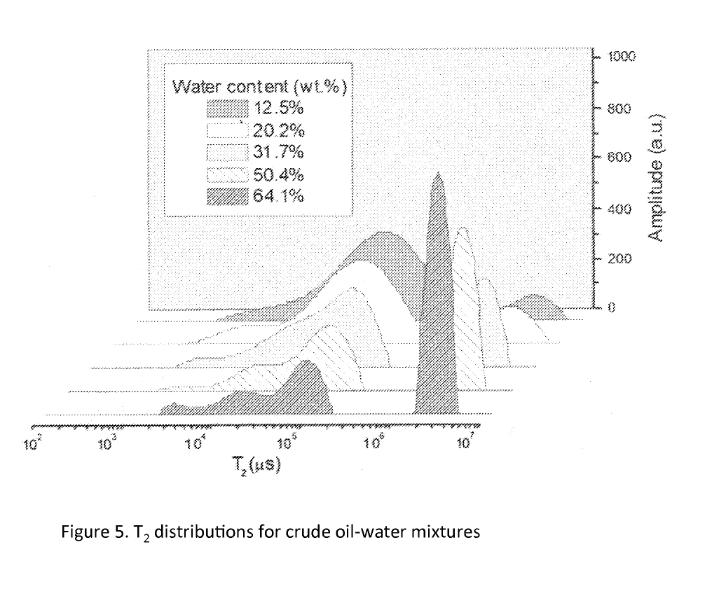

These equations can be inverted to find the “best” set of M_{o,i} subject to constraints, such as all M_{o,i} must be greater than zero. For instance, T₂ distribution curves can be computed from the Inverse Laplace Transform (ILT) of echo data using a logarithmic selection of T₂. FIG. 5 shows some exemplary T₂ distribution curves for oil-water mixture of various concentrations. The water peak around 4 s is evident, which is much shorter than the distribution of T₂ for the crude oil, which occurs between 3 ms and 200 ms.

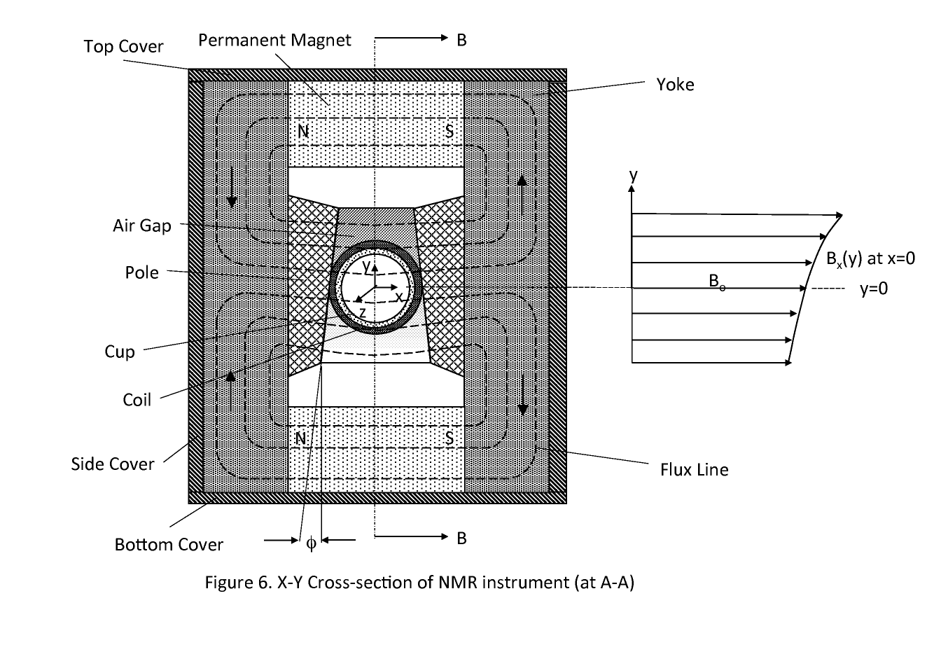

FIG. 6 shows a longitudinal cross-section of an exemplary embodiment of a non-invasive, in-vivo instrument for measuring the constituents of blood. The magneto-motive force (MMF) that generates the static field Bₓ can come from two permanent magnets (PMs). The PMs can be made of rare-earth materials such as neodymium-iron-boron (NdFeB) and/or samarium-cobalt (SmCo), which can have energy-products of approximately 40 MGOe or more. The magnetic flux can be carried by the yokes from the ends of the PMs to the poles. The yokes and/or poles can be made from soft magnetic materials such as carbon steel. Such an arrangement can produce a magnetic field in the air between the poles of approximately 0.6 T. The magnetic materials can be surrounded by a top cover, side covers, bottom cover, and/or front cover made from a non-magnetic material such as aluminum.

The poles can be shaped such that the air gap between the poles varies along the y-axis, as shown in FIG. 6. This can produce a gradient in magnetic field along the y-axis, where the field is greatest where the air gap is least. In FIG. 6, the gradient in the magnetic field is shown by the shading of the air gap. The variation is Bₓ as a function of y is also shown graphically in FIG. 6 at the center of the gap, i.e., at x=0. Alternatively, the spatial variation in Bₓ can be created by slightly skewing one pole face relative to the other by “shimming”.

The static field can pass through a cup that is surrounded by a coil. The static field at the center of the cup (x=0, y=0) is Bₓ(y=0)=Bₒ. The cup can be made of a non-metallic material such as PEEK plastic and/or the coil can be made of copper with very low residual content of iron. When electrical current at radio frequency (RF) flows in the coil, an axial field B₁ can be generated that is in a transverse direction (along the z-axis) to the static field.

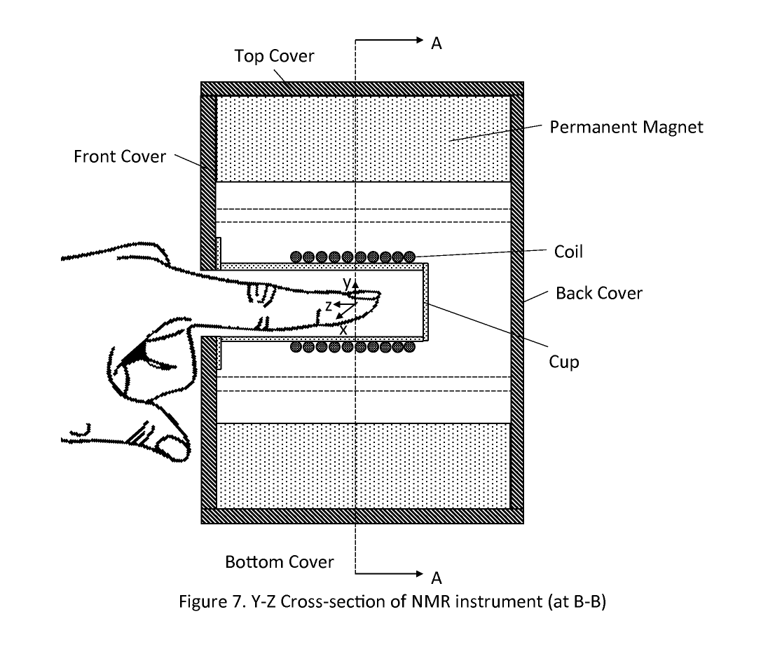

FIG. 7 shows a side cross-section of an exemplary embodiment of a NMR instrument. If a body extremity such as an index finger is positioned in the cup, it can be exposed to the static field Bₓ and/or RF field B₁. Because the static field Bₓ varies with the y-position, the local Larmor frequency also varies with y-position by the relationship

fₒ(y)=γ Bₓ(y)/2π (7)

When the frequency f₁ of the transverse RF field B₁ matches the local value of Larmor frequency fₒ, resonance absorption occurs for the hydrogen nuclei in this location. If the static field is approximately 0.6 T, then the frequency of the transverse field can be approximately 25.5 MHz. Because the absorption occurs primarily for hydrogen nuclei that are in fluids, the NMR signal will tend to be strongest when the RF frequency f₁ matches the Larmor frequency for portions of the finger that contain large amounts of blood. By contrast, the NMR signal will tend to be small when the Larmor frequency is matched in the bone region. As a result, by varying the frequency of the RF field over some range and determining where the maximum signal is generated, the signal-to-noise ratio (SNR) can be increased and/or the ability to discriminate components of blood can be improved. Also, the static field need not be precisely controlled because for each sampling, the frequency of maximum response can be found.

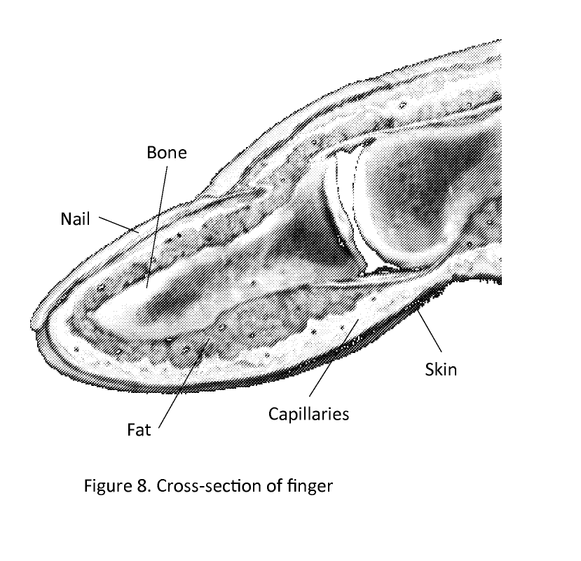

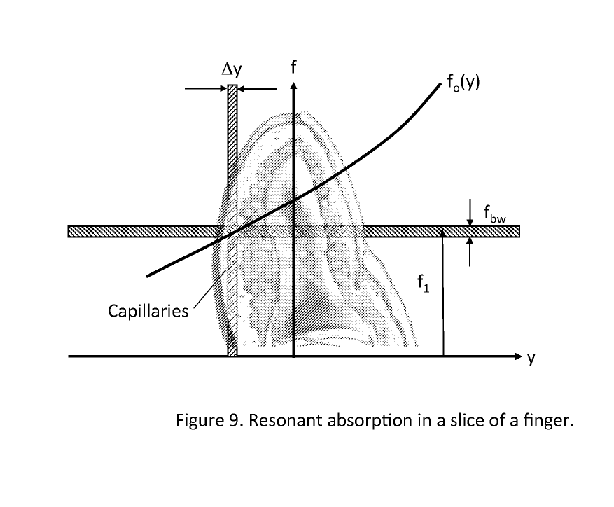

FIG. 8 shows a cross-section of a tip of an exemplary human finger. The bone, fat cells, and capillary features can be clearly seen. Because each feature generally can be exposed to a different static field due to the spatial gradient, the blood-filled capillaries can be found by varying the frequency of the RF field and observing the frequency at which the NMR signal is strongest. This is illustrated in FIG. 9. Because of the gradient in the static magnetic field Bₓ, the Larmor frequency fₒ can vary as a function of the vertical distance y. In FIG. 9, the RF frequency f₁ can be chosen so that the region of the finger that contains capillaries is selected. In fact, the RF signal can be composed of a band of frequencies centered on f₁ but with a bandwidth f_{bw}. The bandwidth can be related to the width of the pulse by the relationship

f_{bw}≈2/τₚ (8)

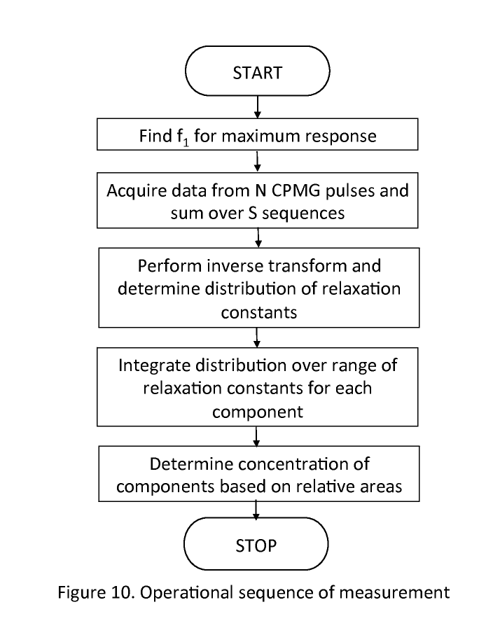

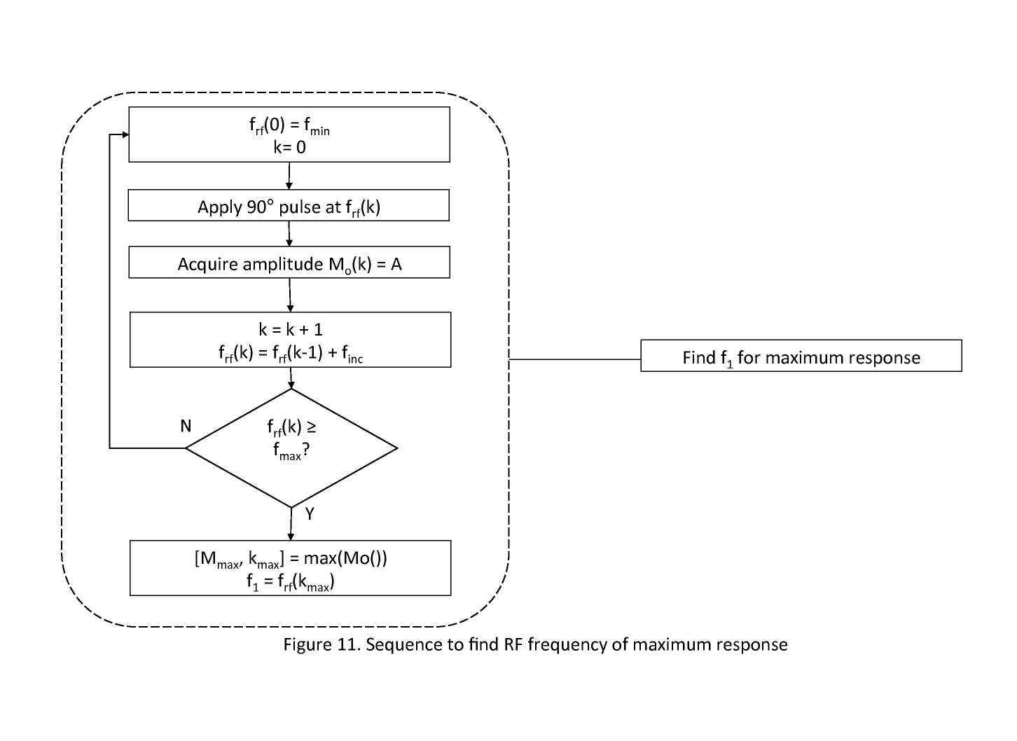

The flowchart for the operation of certain exemplary embodiments is shown in FIG. 10. First, the RF frequency f₁ that gives the maximum response can be determined. An exemplary flowchart to determine this frequency is shown in FIG. 11. The elements of the RF frequency array f_{rf}(k) can comprise values between fₘᵢₙ and fₘₐₓ in increments of f_{inc}. For each f_{rf}(k), a 90-degree pulse can be applied, and/or the corresponding amplitude A of the NMR signal can be recorded as an element in the array Mₒ(k). After the maximum frequency fₘₐₓ is reached and/or the corresponding amplitude is recorded, the array Mₒ(k) can be searched to determine its maximum value Mₘₐₓ and/or the associated index value kₘₐₓ. The corresponding RF frequency therefore can be f_{rF}(kmax) and/or the RF frequency f₁ that is used in the subsequent CPMG spin-echo sequences can be set equal to this value.

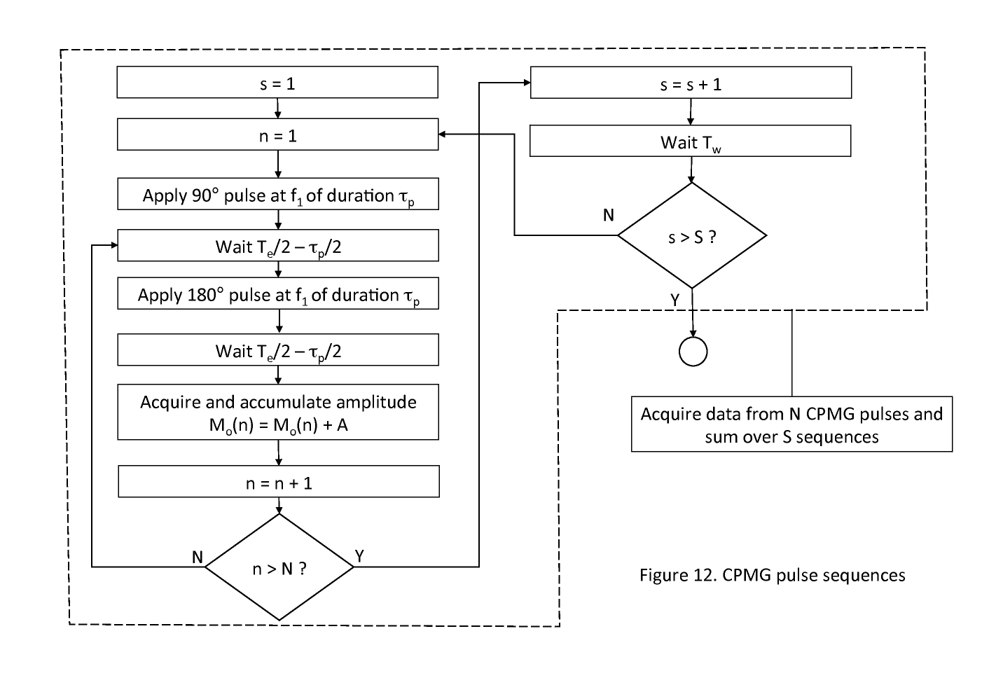

Referring back to FIG. 10, after f₁ is determined, the CPMG sequence can be initiated. An example of this is shown in FIG. 12. A 90-degree pulse can be applied for duration τₚ, followed by a wait of Tₑ-τₚ/2, which then can be followed by a 180-degree pulse of duration τₚ. After a wait of Tₑ-τₚ/2, the amplitude of the echo can be recorded and/or accumulated in an element of the array M(n).The sequence of 180-degree pulses and acquisitions can be repeated N times, and/or with each successive 180-degree pulse, the echo signals can exponentially decay according to the transverse relaxation constant T₂ of each component. Therefore, signals can be acquired at times Tₑ, 2Tₑ, 3Tₑ, . . . nTₑ, . . . NTₑ. Once the signals have decayed to a low value, the train of 180-degree pulses can be stopped and/or a wait period of T_{w} can be established in order to allow sufficient time for the hydrogen nuclei to re-align with applied static field Bₓ. Ideally, this wait period can be greater than several times the value of the spin lattice relaxation time T₁ of any component of interest. The CPMG sequence, followed by a wait period T_{w}, can be repeated S times, and the corresponding values for the decay amplitude at each time nTₑ for every CPMG sequence can be added together to improve the SNR.

Referring back to FIG. 10, once the data for the spin echo decays are obtained, it can be desirable to determine the distribution of relaxation constant T₂. This can be done numerically by finding the inverse transform. The system equations to be solved can be based on an expanded form of Eq. (6):

$$\begin{matrix}M ( 1 ) = {M}_{o , 1} {e}^{- \frac{{T}_{e}}{{T}_{2 , 1}}} + {M}_{o , 2} {e}^{- \frac{{T}_{e}}{{T}_{2 , 2}}} + {M}_{o , 3} {e}^{- \frac{{T}_{e}}{{T}_{2 , 3}}} … + {M}_{o , i} {e}^{- \frac{{T}_{e}}{{T}_{2 , i}}} … + \\ {M}_{o , I} {e}^{- \frac{{T}_{e}}{ {T}_{2 , I}}} \\ M ( 2 ) = {M}_{o , 1} {e}^{- \frac{2 {T}_{e}}{{T}_{2 , 1}}} + {M}_{o , 2} {e}^{- \frac{2 {T}_{e}}{{T}_{2 , 2}}} + {M}_{o , 3} {e}^{- \frac{2 {T}_{e}}{{T}_{2 , 3}}} … + {M}_{o , i} {e}^{- \frac{2 {T}_{e}}{{T}_{2 , i}}} … + \\ {M}_{o , I} {e}^{- \frac{2 {T}_{e}}{ {T}_{2 , I}}} \\ M ( 3 ) = {M}_{o , 1} {e}^{- \frac{3 {T}_{e}}{{T}_{2 , 1}}} + {M}_{o , 2} {e}^{- \frac{3 {T}_{e}}{{T}_{2 , 2}}} + {M}_{o , 3} {e}^{- \frac{3 {T}_{e}}{{T}_{2 , 3}}} … + {M}_{o , i} {e}^{- \frac{3 {T}_{e}}{{T}_{2 , i}}} … + \\ {M}_{o , I} {e}^{- \frac{3 {T}_{e}}{ {T}_{2 , I}}} \\ • \\ • \\ • \\ M ( n ) = {M}_{o , 1} {e}^{- \frac{{nT}_{e}}{{T}_{2 , 1}}} + {M}_{o , 2} {e}^{- \frac{{nT}_{e}}{{T}_{2 , 2}}} + {M}_{o , 3} {e}^{- \frac{{nT}_{e}}{{T}_{2 , 3}}} … + {M}_{o , i} {e}^{- \frac{{nT}_{e}}{{T}_{2 , i}}} … + \\ {M}_{o , I} {e}^{- \frac{{nT}_{e}}{ {T}_{2 , I}}} \\ • \\ • \\ • \\ M ( N ) = {M}_{o , 1} {e}^{- \frac{{NT}_{e}}{{T}_{2 , 1}}} + {M}_{o , 2} {e}^{- \frac{{NT}_{e}}{{T}_{2 , 2}}} + {M}_{o , 3} {e}^{- \frac{{NT}_{e}}{{T}_{2 , 3}}} … + {M}_{o , i} {e}^{- \frac{{NT}_{e}}{{T}_{2 , i}}} … + \\ {M}_{o , I} {e}^{- \frac{{NT}_{e}}{ {T}_{2 , I}}}\end{matrix}$$

In this system equations, the unknowns are the initial magnetization value M_{o,i} for each component i, for a total of I unknowns. The values of T_{2,i} can be assumed to be known by assigning a distribution of values of T_{2,i} in the sample. For instance, a geometric progression can be chosen such as 1, 2, 4, 8, . . . , 8192 ms. The number of equations is N, which represents the number of times the 180-degree pulse is applied and data is acquired for each CPMG sequence. In general, N is greater than 1, representing the number of components. To solve these equations, techniques such as the Inverse Laplace Transform (ILT) can be employed, or the values of M_{o,i} can be determined iteratively using Least Square Errors techniques.

Referring back to FIG. 10, once the distribution of relaxation constants is obtained, we can use this distribution to determine the relative concentrations of blood constituents. Each blood constituent, e.g., water, glucose, and/or cholesterol, can be represented in the distribution as a range of T_{2,i} between a minimum and maximum value. By summing the values of the distribution M_{o,i} over this range, the relative magnitude of the NMR magnetization due to that blood constituent can be determined. The relative concentration in the blood then can be determined to be proportional to the ratio of the NMR signal for that constituent compared to the NMR signal corresponding to water.

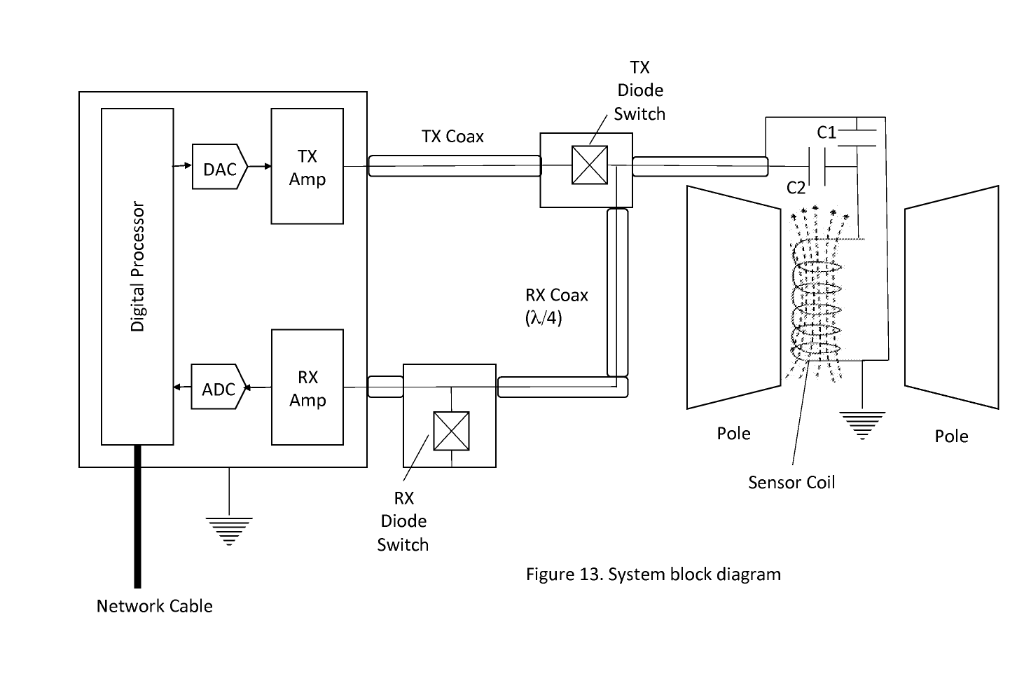

FIG. 13 shows a block diagram of an exemplary NMR instrument. A digital processor can control the timing, frequency, and/or amplitude of the RF pulses that ultimately can be sent to the sensor coil. The RF pulse output of the digital processor can be fed to a power amplifier, which in turn can be connected by the RF coaxial cable to a transmit (TX) diode switch. The TX diode switch can pass the RF signals to the sensor during transmit (pulse generation) and/or can isolate the receive circuitry from the transmit circuitry when acquiring data. Capacitor C₁ can be electrically in parallel with the sensor coil and/or can be electrically in series with capacitor C₂. The values of C₁ and C₂ can be chosen so that the inductance Lₛ of the coil is cancelled and/or the resistance Rₛ of the coil is transformed to a standard impedance such as approximately 50 ohms, which can be the characteristic impedance of the coaxial cable, the output impedance of the TX amplifier, and/or the input impedance of the receive (RX) amplifier. The RX diode switch can pass the signals from the sensor to the RX amplifier when acquiring data. The RX diode switch, in combination with the quarter-wave (¼) coaxial cable, can ensure that no damage occurs to the RX amplifier circuitry when RF pulses are generated.

The digital processor can be connected to a data network via wired and/or wireless connection. Data acquired by the NMR instrument can be sent to a remote location via a network, such as the Internet, a local area network, and/or other network system, where that data can be analyzed and/or stored as appropriate. This remote analysis and/or storage can be particularly convenient if the NMR device is located in a home or small medical office and no medical personnel having the appropriate training are available at this location.

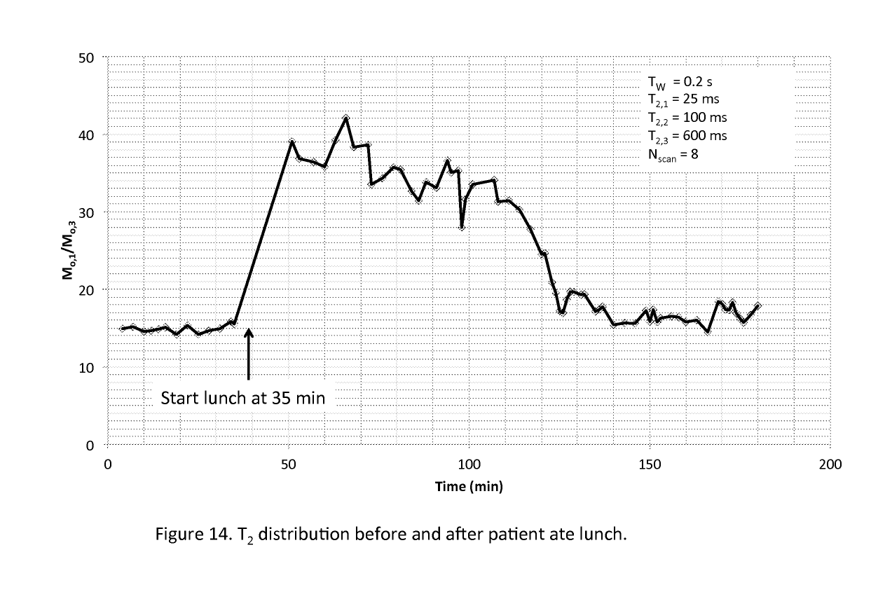

FIG. 14 is a plot of exemplary data that was obtained from a patient before and after eating lunch. The exponential decays from the CPMG sequences were fit to a three-group model for which T_{2,1}=25 ms, T_{2,2}=100 ms, and T_{2,3}=600 ms. The static field was about 0.34 T, corresponding to a Larmor frequency of about 14.5 MHz, and at each time 8 scans were performed with a repetition time of Tᵣₑₚ=0.2 s. At 35 minutes into the test, the patient ate lunch and data collection resumed 16 minutes later at 51 minutes.

The ratio M_{o,1}/M_{o,3} was plotted versus time, where M_{o,1} represents the concentration of species with a T₂ relaxation rate of 25 ms and M_{o,3} represents the concentration of species with a T₂ relaxation rate of 600 ms. After lunch, there was a clear increase in this ratio from a baseline value of about 15 to a peak value of about 42 which occurred at about 30 minutes after the meal. Within 105 minutes after eating, baseline levels had returned. Subsequent testing of this patient's blood glucose level using an off-the-shelf blood glucose monitoring system indicated a baseline value of about 80 mg/dL and a typical post-prandial level of about 130 mg/dL.

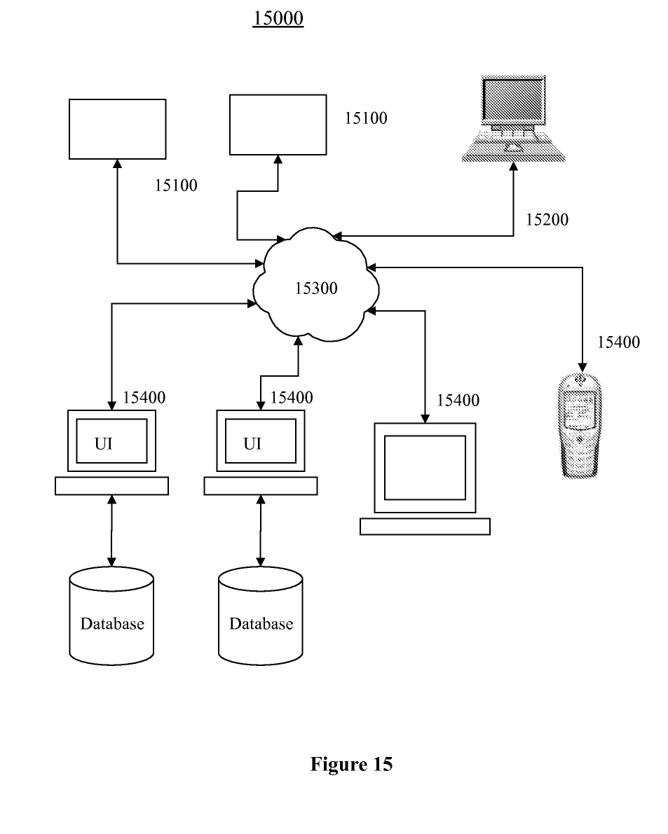

FIG. 15 is a block diagram of an exemplary embodiment of a system 15000, which can comprise one or more NMR instruments 15100 that can be communicatively coupled to a local information device 15200 and/or a network 15300 to which one or more remote information devices 15400 (e.g., desktop computers, laptop computers, tablet computers, smart phones, and/or servers, etc.) can be communicatively coupled. Any information device can host NMR analysis software and/or a data repository for data related to NMR and/or blood components etc.

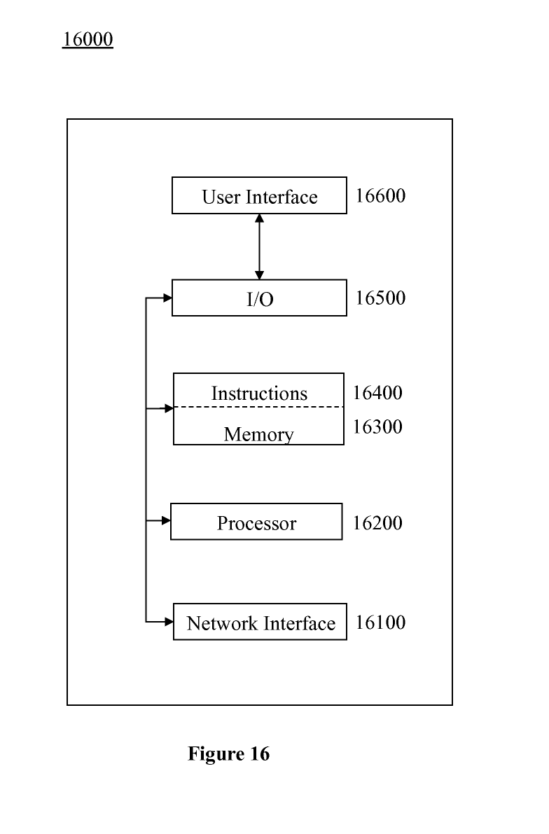

FIG. 16 is a block diagram of an exemplary embodiment of an information device 16000, which in certain operative embodiments can comprise, for example, and information device of FIG. 15. Information device 16000 can comprise any of numerous transform circuits, which can be formed via any of numerous communicatively-, electrically-, magnetically-, optically-, fluidically-, and/or mechanically-coupled physical components, such as for example, one or more network interfaces 16100, one or more processors 16200, one or more memories 16300 containing instructions 16400, one or more input/output (I/O) devices 16500, and/or one or more user interfaces 16600 coupled to I/O device 16500, etc.

In certain exemplary embodiments, via one or more user interfaces 16600, such as a graphical user interface, a user can view a rendering of information related to researching, designing, modeling, creating, developing, building, manufacturing, operating, maintaining, storing, marketing, selling, delivering, selecting, specifying, requesting, ordering, receiving, returning, rating, and/or recommending any of the products, services, methods, user interfaces, and/or information described herein.

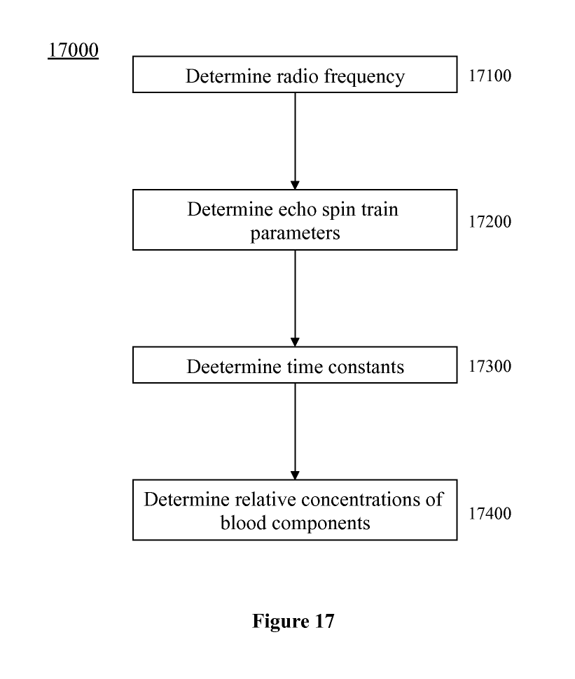

FIG. 17 is a flowchart of an exemplary embodiment of a method 17000. At activity 17100, an desired radio frequency can be determined. At activity 17200, the radio frequency can be applied to determine parameters (e.g., amplitude, spin-spin relaxation time, etc.) of an echo spin train. At activity 17300, a spin-spin relaxation time constant distribution can be determined. At activity 17400, a relative concentration of blood components can be determined.

Certain exemplary embodiments can provide:
An NMR device adapted for the in-vivo measurement of blood constituents by determining the distribution of relaxation constants from NMR echo trains in order to determine relative concentrations of blood constituents such as glucose, cholesterol, and alcohol;
An NMR device adapted to improve the signal-to-noise ratio for the in-vivo measurement of blood components by positioning a body extremity in a static magnetic field with a gradient and varying the RF frequency of a transverse magnetic field to find the frequency which results in the maximum response;
Communicatively coupling an NMR device over a cable or wire-less network for remote analysis and/or storage; and/or
An NMR device that can be readily human-portable, which can be conducive to measuring blood components such as glucose, alcohol, and/or cholesterol, by, for example, patients at home or while traveling, emergency responders, police officers, mobile medical personnel, medical staff at small clinics, etc.

Certain exemplary embodiments can provide a method comprising:
via one or more predetermined processors communicatively coupled to sensor coil of a nuclear magnetic resonance instrument:
   determining a radio frequency that substantially matches a hydrogen nuclei Larmor frequency for a capillary-rich portion of a digit of a mammal, the capillary-rich portion containing a large amount of blood relative to a bone portion of the digit, the hydrogen nuclei Larmor frequency corresponding to a static magnetic field induced by one or more permanent magnets to cross an air gap between an opposing pair of pole faces that have a transverse spacing sufficient to receive a cup that is configured to receive the digit, a magnitude of the hydrogen nuclei Larmor frequency dependent on a position of the portion of the digit between the pair of pole faces, the radio frequency a measure of time-dependent variation in a longitudinal magnetic field induced by a time-varying current in the sensor coil, the sensor coil substantially surrounding the cup and defining a coil axis oriented substantially parallel to a longitudinal axis of the cup;
   while the longitudinal magnetic field is applied to the digit, acquiring an amplitude and a spin-spin relaxation time of each of a train of spin echoes created by applying a plurality of CPMG pulses to the digit via the sensor coil, a count of the spin echoes in the train of spin echoes corresponding to a decay of the spin echo amplitudes to a predetermined value;
   based on the amplitudes of the spin echoes, determining a distribution of spin-spin relaxation time constants of a plurality of components in the blood;
   for each of the one or more predetermined components, based on the distribution of spin-spin relaxation time constants, determining a relative concentration of the predetermined component in the blood;
   repeating said acquiring for a predetermined number of repetitions;
   repeating said acquiring for a predetermined number of repetitions, each repetition delayed by a wait time that is greater than a spin lattice relaxation time of one or more predetermined components of the plurality of components;
   repeating said acquiring for a predetermined number of repetitions, each repetition delayed by a wait time that is less than a spin lattice relaxation time of one or more predetermined components of the plurality of components;
   repeating said acquiring for a predetermined number (N) of repetitions such that a plurality of echo trains is acquired, each echo train comprising a plurality echoes, each echo from each echo train having a corresponding sequential position in that echo train;
   for the plurality of echo trains, for each sequential position, summing an amplitude of the corresponding echoes, such that all first echoes are summed together, all second echoes are summed together, and all N echoes are summed together;
   repeating said acquiring for a predetermined number of repetitions;
   summing similarly timed echoes across the predetermined number of repetitions; and/or
   rendering the relative concentration of the one or more predetermined component in the blood;
      wherein:
at least one of the one or more processors is communicatively coupled to the sensor coil via a network.

Certain exemplary embodiments can provide a method comprising:
via one or more predetermined processors communicatively coupled to sensor coil of a nuclear magnetic resonance instrument:
   for each of one or more predetermined components of a plurality of components in blood of a digit of a mammal, based on a distribution of spin-spin relaxation time constants for hydrogen nuclei of the predetermined component, determining a relative concentration of the predetermined component in the blood, the distribution of spin-spin relaxation time constants determined based on amplitudes of a train of spin echoes created by a plurality of CPMG pulses applied to the digit by a sensor coil while a longitudinal magnetic field is applied to the digit, a count of the spin echoes in the train of spin echoes corresponding to decay of the spin echoes to a predetermined value, the sensor coil substantially surrounding a cup and defining a coil axis oriented substantially parallel to a longitudinal axis of the cup, the cup configured to receive the digit, the cup located within a transverse spacing between an opposing pair of pole faces of one or more permanent magnets, the transverse spacing defining an air gap across which the one or more permanent magnets are configured to produce a static magnetic field, the static magnetic field configured to induce hydrogen nuclei of the digit to precess at a corresponding Larmor frequency, the Larmor frequency of each hydrogen nuclei having a magnitude that is dependent on a position of a portion of the digit between the pair of pole faces, a time-dependent variation in the longitudinal magnetic field applied by a time-varying current in the sensor coil having a frequency substantially matching the Larmor frequency for a capillary-rich portion of the digit, the capillary-rich portion containing a large amount of blood relative to a bone portion of the digit.

Certain exemplary embodiments can provide a method comprising:
via one or more predetermined processors communicatively coupled to sensor coil of a nuclear magnetic resonance instrument:
   determining a radio frequency that substantially matches a hydrogen nuclei Larmor frequency for a capillary-rich portion of a digit of a mammal, the capillary-rich portion containing a large amount of blood relative to a bone portion of the digit, the hydrogen nuclei Larmor frequency corresponding to a static magnetic field induced one or more permanent magnets to cross an air gap between an opposing pair of pole faces that have a transverse spacing sufficient to receive a cup that is configured to receive the digit, a magnitude of the hydrogen nuclei Larmor frequency dependent on a position of the portion of the digit between the pair of pole faces, the radio frequency a measure of time-dependent variation in a longitudinal magnetic field induced by a time-varying current in the sensor coil, the sensor coil substantially surrounding the cup and defining a coil axis oriented substantially parallel to a longitudinal axis of the cup.

Certain exemplary embodiments can provide a device comprising:
a cup configured to receive at least a terminal portion of a digit of a mammal, the cup defining a cup longitudinal axis;
one or more permanent magnets configured to induce a static magnetic field to cross an air gap located between an opposing pair of pole faces that have a transverse spacing sufficient to receive the cup;
a sensor coil substantially surrounding the cup, defining a coil axis oriented substantially parallel to a longitudinal axis of the cup, and configured to produce a longitudinal magnetic field that varies with respect to time responsive to application of a time-varying current to the sensor coil;
wherein:
   the static magnetic field is configured to induce hydrogen nuclei of the digit to precess at a corresponding Larmor frequency;
   the Larmor frequency of each hydrogen nuclei has a magnitude that is dependent on a position of a portion of the digit between the pair of pole faces;
   the time-dependent variation in the longitudinal magnetic field has a frequency substantially matching a Larmor frequency for a capillary-rich portion of the digit, the capillary-rich portion containing a large amount of blood relative to a bone portion of the digit.

Certain exemplary embodiments can provide a device comprising:
a cup configured to receive at least a terminal portion of a digit of a mammal, the cup defining a cup longitudinal axis;
one or more permanent magnets configured to induce a static magnetic field to cross an air gap located between an opposing pair of pole faces that have a transverse spacing sufficient to receive the cup; and/or
a sensor coil substantially surrounding the cup, defining a coil axis oriented substantially parallel to a longitudinal axis of the cup, and configured to produce a longitudinal magnetic field that varies with respect to time responsive to application of a time-varying current to the sensor coil;
wherein:
   the sensor coil is configured to acquire a train of spin echoes created by a plurality of CPMG pulses applied to the digit by a sensor coil while the longitudinal magnetic field is applied to the digit, the train of spin echoes defining amplitudes and corresponding spin-spin relaxation times, the amplitudes and spin-spin relaxation times corresponding to a distribution of spin-spin relaxation time constants for hydrogen nuclei of a predetermined component of a plurality of components of blood of the mammal, the distribution corresponding to a relative concentration of the predetermined component in the blood.

### Definitions

When the following terms are used substantively herein, the accompanying definitions apply. These terms and definitions are presented without prejudice, and, consistent with the application, the right to redefine these terms via amendment during the prosecution of this application or any application claiming priority hereto is reserved. For the purpose of interpreting a claim of any patent that claims priority hereto, each definition in that patent functions as a clear and unambiguous disavowal of the subject matter outside of that definition.
a—at least one.
about—around and/or approximately.
above—at a higher level.
acquire—to obtain and/or gain possession of
across—from one side to another.
activity—an action, act, step, and/or process or portion thereof.
adapt—to design, make, set up, arrange, shape, configure, and/or make suitable and/or fit for a specific purpose, function, use, and/or situation.
adapted to—suitable, fit, and/or capable of performing a specified function.
after—following in time and/or subsequent to.
air—the earth's atmospheric gas.
all—every.
along—through, on, beside, over, in line with, and/or parallel to the length and/or direction of; and/or from one end to the other of.
amount—a quantity.
amplitude—a magnitude of a variable.
an—at least one.
and—in conjuction with.
and/or—either in conjunction with or in alternative to.
any—one, some, every, and/or all without specification.
apparatus—an appliance and/or device for a particular purpose.
application—the act of putting something to a use and/or purpose; and/or using something for a particular purpose.
applied—incident directly and/or indirectly upon.
apply—to put to, on, and/or into action and/or service; to implement; and/or to bring into contact with something.
approximately—about and/or nearly the same as.
are—to exist.
around—about, surrounding, and/or on substantially all sides of; and/or approximately.
as long as—if and/or since.
associate—to join, connect together, accompany, and/or relate.
at—in, on, and/or near.
at least—not less than, and possibly more than.
at least one—not less than one, and possibly more than one.
automatic—performed via an information device in a manner essentially independent of influence and/or control by a user. For example, an automatic light switch can turn on upon “seeing” a person in its “view”, without the person manually operating the light switch.
axis—a straight line about which a body and/or geometric object rotates and/or can be conceived to rotate and/or a center line to which parts of a structure and/or body can be referred.
based—being derived from, conditional upon, and/or dependent upon.
between—in a separating interval and/or intermediate to.
blood—a fluid consisting of plasma, blood cells, and platelets that is circulated by the heart through the vertebrate vascular system, carrying oxygen and nutrients to and waste materials away from all body tissues.
bone—a dense, semirigid, porous, calcified connective tissue forming the major portion of the skeleton of most vertebrates and constructed of a dense organic matrix and an inorganic, mineral component.
Boolean logic—a complete system for logical operations.
by—via and/or with the use and/or help of.
can—is capable of, in at least some embodiments.
capillary—one of the minute blood vessels between the terminations of the arteries and the beginnings of the veins.
cause—to bring about, provoke, precipitate, produce, elicit, be the reason for, result in, and/or effect.
circuit—a physical system comprising, depending on context: an electrically conductive pathway, an information transmission mechanism, and/or a communications connection, the pathway, mechanism, and/or connection established via a switching device (such as a switch, relay, transistor, and/or logic gate, etc.); and/or an electrically conductive pathway, an information transmission mechanism, and/or a communications connection, the pathway, mechanism, and/or connection established across two or more switching devices comprised by a network and between corresponding end systems connected to, but not comprised by the network.
coil—(n) a continuous loop comprising two or more turns of electrically conductive material. (v) to roll and/or form into a configuration having a substantially spiraled cross-section.
coil axis—that path along which a unit magnetic pole would experience a maximum force when a current is caused to flow in the coil conductor. For example, in a long, uniform, single layer cylindrical coil, the coil axis corresponds to the geometrical axis of the coil.
communicatively—linking in a manner that facilitates communications.
component—a constituent element and/or part.
composition of matter—a combination, reaction product, compound, mixture, formulation, material, and/or composite formed by a human and/or automation from two or more substances and/or elements.
compound—a pure, macroscopically homogeneous substance consisting of atoms or ions of two or more different elements in definite proportions that cannot be separated by physical methods. A compound usually has properties unlike those of its constituent elements.
comprising—including but not limited to.
concentration—a measure of the amount of dissolved substance contained per unit of volume and/or the amount of a specified substance in a unit amount of another substance.
configure—to design, arrange, set up, shape, and/or make suitable and/or fit for a specific purpose, function, use, and/or situation.
connect—to join or fasten together.
containing—including but not limited to.
convert—to transform, adapt, and/or change.
corresponding—related, associated, accompanying, similar in purpose and/or position, conforming in every respect, and/or equivalent and/or agreeing in amount, quantity, magnitude, quality, and/or degree.
count—(n.) a number reached by counting and/or a defined quantity; (v.) to increment, typically by one and beginning at zero.
coupleable—capable of being joined, connected, and/or linked together.
coupled—connected or linked by any known means, including mechanical, fluidic, acoustic, electrical, magnetic, and/or optical, etc.
create—to make, form, produce, generate, bring into being, and/or cause to exist.
cross—to go and/or extend across, pass from one side of to the other, carry and/or conduct across something, and/or extend and/or pass through and/or over.
cup—a tube having one end closed.
current—a flow of electrical energy.
data—distinct pieces of information, usually formatted in a special or predetermined way and/or organized to express concepts, and/or represented in a form suitable for processing by an information device.
data structure—an organization of a collection of data that allows the data to be manipulated effectively and/or a logical relationship among data elements that is designed to support specific data manipulation functions. A data structure can comprise meta data to describe the properties of the data structure. Examples of data structures can include: array, dictionary, graph, hash, heap, linked list, matrix, object, queue, ring, stack, tree, and/or vector.
decay—(v) to decrease gradually in magnitude; (n) a gradual deterioration to a different, lower, and/or an inferior state.
define—to establish the meaning, relationship, outline, form, and/or structure of; and/or to precisely and/or distinctly describe and/or specify.
delay—an elapsed time between two states and/or events.
dependent—relying upon and/or contingent upon.
derive—to receive, obtain, and/or produce from a source and/or origin.
determine—to find out, obtain, calculate, decide, deduce, ascertain, and/or come to a decision, typically by investigation, reasoning, and/or calculation.
determined—found and/or decided upon.
device—a machine, manufacture, and/or collection thereof.
digit—any of the divisions (such as a finger or toe) in which the limbs of amphibians and all higher vertebrates including humans terminate, which are typically five in number but may be reduced (as in the horse), and which typically have a series of phalanges bearing a nail, claw, or hoof at the tip.
digital—non-analog and/or discrete.
distribution—a set of data, events, occurrences, outcomes, objects, and/or entities and their frequency of occurrence collected from measurements over a statistical population.
each—every one of a group considered individually.
effective—sufficient to bring about, provoke, elicit, and/or cause.
embodiment—an implementation, manifestation, and/or a concrete representation, such as of a concept.
estimate—(n) a calculated value approximating an actual value; (v) to calculate and/or determine approximately and/or tentatively.
exemplary—serving as an example, model, instance, and/or illustration.
face—the most significant or prominent surface of an object.
first—an initial entity in an ordering of entities and/or immediately preceding the second in an ordering.
for—with a purpose of.
frequency—a number of times a specified periodic phenomenon occurs within a specified interval, and/or a number of complete alternations or cycles made by an alternating signal per unit of time. The frequency unit most used is cycles per second.
from—used to indicate a source, origin, and/or location thereof.
further—in addition.
gap—a space between objects.
generate—to create, produce, render, give rise to, and/or bring into existence.
greater than—larger and/or more than.
haptic—involving the human sense of kinesthetic movement and/or the human sense of touch. Among the many potential haptic experiences are numerous sensations, body-positional differences in sensations, and time-based changes in sensations that are perceived at least partially in non-visual, non-audible, and non-olfactory manners, including the experiences of tactile touch (being touched), active touch, grasping, pressure, friction, traction, slip, stretch, force, torque, impact, puncture, vibration, motion, acceleration, jerk, pulse, orientation, limb position, gravity, texture, gap, recess, viscosity, pain, itch, moisture, temperature, thermal conductivity, and thermal capacity.
have—to possess as a characteristic, quality, or function.
having—possessing, characterized by, comprising, and/or including, but not limited to.
human-machine interface—hardware and/or software adapted to render information to a user and/or receive information from the user; and/or a user interface.
hydrogen—an element defined by each atom comprising a single proton and a single electron.
including—having, but not limited to, what follows.
induce—to bring about and/or cause to occur.
information device—any device capable of processing data and/or information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as an iPhone-like and/or Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can comprise components such as one or more network interfaces, one or more processors, one or more memories containing instructions, and/or one or more input/output (I/O) devices, one or more user interfaces coupled to an I/O device, etc.
initialize—to prepare something for use and/or some future event.
input/output (I/O) device—any device adapted to provide input to, and/or receive output from, an information device. Examples can include an audio, visual, haptic, olfactory, and/or taste-oriented device, including, for example, a monitor, display, projector, overhead display, keyboard, keypad, mouse, trackball, joystick, gamepad, wheel, touchpad, touch panel, pointing device, microphone, speaker, video camera, camera, scanner, printer, switch, relay, haptic device, vibrator, tactile simulator, and/or tactile pad, potentially including a port to which an I/O device can be attached or connected.
install—to connect or set in position and prepare for use.
instructions—directions, which can be implemented as hardware, firmware, and/or software, the directions adapted to perform a particular operation and/or function via creation and/or maintenance of a predetermined physical circuit.
instrument—a device for recording, measuring, or controlling, especially such a device functioning as part of a control system.
into—to a condition, state, or form of.
is—to exist in actuality.
larger—great in magnitude.
Larmor frequency—a rate of precession of a magnetic moment of a nucleus around an external magnetic field.
less than—having a measurably smaller magnitude and/or degree as compared to something else.
located—situated approximately in a particular spot and/or position.
logic gate—a physical device adapted to perform a logical operation on one or more logic inputs and to produce a single logic output, which is manifested physically. Because the output is also a logic-level value, an output of one logic gate can connect to the input of one or more other logic gates, and via such combinations, complex operations can be performed. The logic normally performed is Boolean logic and is most commonly found in digital circuits. The most common implementations of logic gates are based on electronics using resistors, transistors, and/or diodes, and such implementations often appear in large arrays in the form of integrated circuits (a.k.a., IC's, microcircuits, microchips, silicon chips, and/or chips). It is possible, however, to create logic gates that operate based on vacuum tubes, electromagnetics (e.g., relays), mechanics (e.g., gears), fluidics, optics, chemical reactions, and/or DNA, including on a molecular scale. Each electronically-implemented logic gate typically has two inputs and one output, each having a logic level or state typically physically represented by a voltage. At any given moment, every terminal is in one of the two binary logic states (“false” (a.k.a., “low” or “0”) or “true” (a.k.a., “high” or “1”), represented by different voltage levels, yet the logic state of a terminal can, and generally does, change often, as the circuit processes data. . Thus, each electronic logic gate typically requires power so that it can source and/or sink currents to achieve the correct output voltage. Typically, machine-implementable instructions are ultimately encoded into binary values of “0”s and/or “1”s and, are typically written into and/or onto a memory device, such as a “register”, which records the binary value as a change in a physical property of the memory device, such as a change in voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc. An exemplary register might store a value of “01101100”, which encodes a total of 8 “bits” (one byte), where each value of either “0” or “1” is called a “bit” (and 8 bits are collectively called a “byte”). Note that because a binary bit can only have one of two different values (either “0” or “1”), any physical medium capable of switching between two saturated states can be used to represent a bit. Therefore, any physical system capable of representing binary bits is able to represent numerical quantities, and potentially can manipulate those numbers via particular encoded machine-implementable instructions. This is one of the basic concepts underlying digital computing. At the register and/or gate level, a computer does not treat these “0”s and “1”s as numbers per se, but typically as voltage levels (in the case of an electronically-implemented computer), for example, a high voltage of approximately +3 volts might represent a “1” or “logical true” and a low voltage of approximately 0 volts might represent a “0” or “logical false” (or vice versa, depending on how the circuitry is designed). These high and low voltages (or other physical properties, depending on the nature of the implementation) are typically fed into a series of logic gates, which in turn, through the correct logic design, produce the physical and logical results specified by the particular encoded machine-implementable instructions. For example, if the encoding request a calculation, the logic gates might add the first two bits of the encoding together, produce a result “1” (“0”+“1”=“1”), and then write this result into another register for subsequent retrieval and reading. Or, if the encoding is a request for some kind of service, the logic gates might in turn access or write into some other registers which would in turn trigger other logic gates to initiate the requested service.
logical—a conceptual representation.
longitudinal—of and/or relating to a length; placed and/or running lengthwise.
longitudinal axis—a straight line defined parallel to an object's length and passing through a centroid of the object.
machine-implementable instructions—directions adapted to cause a machine, such as an information device, to perform one or more particular activities, operations, and/or functions via forming a particular physical circuit. The directions, which can sometimes form an entity called a “processor”, “kernel”, “operating system”, “program”, “application”, “utility”, “subroutine”, “script”, “macro”, “file”, “project”, “module”, “library”, “class”, and/or “object”, etc., can be embodied and/or encoded as machine code, source code, object code, compiled code, assembled code, interpretable code, and/or executable code, etc., in hardware, firmware, and/or software.
machine-readable medium—a physical structure from which a machine, such as an information device, computer, microprocessor, and/or controller, etc., can store one or more machine-implementable instructions, data, and/or information and/or obtain one or more stored machine-implementable instructions, data, and/or information. Examples include a memory device, punch card, player-plano scroll, etc.
magnet—a body that can attract certain substances, such as iron or steel, as a result of a magnetic field
magnetic—having the property of attracting iron and certain other materials by virtue of a surrounding field of force.
magnetic field—a the portion of space near a magnetic body or a current-carrying body in which the magnetic forces due to the body or current can be detected.
magnitude—a size and/or extent.

mammal—Any of various warm-blooded vertebrate animals of the class Mammalia, including humans, characterized by a covering of hair on the skin and, in the female, milk-producing mammary glands for nourishing the young.
match—(n) one that fits, meets, resembles, harmonizes, find a counterpart to, and/or corresponds in one or more attributes. (v) to mirror, resemble, harmonize, fit, correspond, and/or determine a correspondence between, two or more values, entities, and/or groups of entities.
may—is allowed and/or permitted to, in at least some embodiments.
measure—(n) a quantity ascertained by comparison with a standard and/or manual and/or automatic observation. (v) to physically sense, and/or determine a value and/or quantity of something relative to a standard.
memory device—an apparatus capable of storing, sometimes permanently, machine-implementable instructions, data, and/or information, in analog and/or digital format. Examples include at least one non-volatile memory, volatile memory, register, relay, switch, Random Access Memory, RAM, Read Only Memory, ROM, flash memory, magnetic media, hard disk, floppy disk, magnetic tape, optical media, optical disk, compact disk, CD, digital versatile disk, DVD, and/or raid array, etc. The memory device can be coupled to a processor and/or can store and provide instructions adapted to be executed by processor, such as according to an embodiment disclosed herein.
method—one or more acts that are performed upon subject matter to be transformed to a different state or thing and/or are tied to a particular apparatus, said one or more acts not a fundamental principal and not pre-empting all uses of a fundamental principal.
more—a quantifier meaning greater in size, amount, extent, and/or degree.
near—a distance of less than approximately [X].
network—a communicatively coupled plurality of nodes, communication devices, and/or information devices. Via a network, such nodes and/or devices can be linked, such as via various wireline and/or wireless media, such as cables, telephone lines, power lines, optical fibers, radio waves, and/or light beams, etc., to share resources (such as printers and/or memory devices), exchange files, and/or allow electronic communications therebetween. A network can be and/or can utilize any of a wide variety of sub-networks and/or protocols, such as a circuit switched, public-switched, packet switched, connection-less, wireless, virtual, radio, data, telephone, twisted pair, POTS, non-POTS, DSL, cellular, telecommunications, video distribution, cable, radio, terrestrial, microwave, broadcast, satellite, broadband, corporate, global, national, regional, wide area, backbone, packet-switched TCP/IP, IEEE 802.03, Ethernet, Fast Ethernet, Token Ring, local area, wide area, IP, public Internet, intranet, private, ATM, Ultra Wide Band (UWB), Wi-Fi, BlueTooth, Airport, IEEE 802.11, IEEE 802.11a, IEEE 802.11b, IEEE 802.11g, X-10, electrical power, 3G, 4G, multi-domain, and/or multi-zone sub-network and/or protocol, one or more Internet service providers, one or more network interfaces, and/or one or more information devices, such as a switch, router, and/or gateway not directly connected to a local area network, etc., and/or any equivalents thereof.
network interface—any physical and/or logical device, system, and/or process capable of coupling an information device to a network. Exemplary network interfaces comprise a telephone, cellular phone, cellular modem, telephone data modem, fax modem, wireless transceiver, communications port, Ethernet card, cable modem, digital subscriber line interface, bridge, hub, router, or other similar device, software to manage such a device, and/or software to provide a function of such a device.
no—an absence of and/or lacking any.
nuclear magnetic resonance—an absorption of electromagnetic radiation of a specific frequency by an atomic nucleus that is placed in a relatively strong magnetic field; and/or an absorption of electromagnetic energy (typically radio waves) by the nuclei of atoms placed in a strong magnetic field, whereby nuclei of different atoms absorb unique frequencies of radiation depending on their environment, thus by observing which frequencies are absorbed by a sample placed in a strong magnetic field (and later emitted again, when the magnetic field is removed), it is possible to learn much about the sample's makeup and structure.
nuclei—a plural of nucleus.
nucleus—the positively charged central region of an atom, composed of protons and neutrons and containing almost all of the mass of the atom.
number—a count and/or quantity.
one—being and/or amounting to a single unit, individual, and/or entire thing, item, and/or object.
operable—practicable and/or fit, ready, and/or configured to be put into its intended use and/or service.
opposing—opposite; against; being the other of two complementary or mutually exclusive things; and/or placed or located opposite, in contrast, in counterbalance, and/or across from something else and/or from each other.
or—a conjunction used to indicate alternatives, typically appearing only before the last item in a group of alternative items.
orient—to position a first object relative to a second object.
outside—beyond a range, boundary, and/or limit; and/or not within.
packet—a generic term for a bundle of data organized in a specific way for transmission, such as within and/or across a network, such as a digital packet-switching network, and comprising the data to be transmitted and certain control information, such as a destination address.
pair—a quantity of two of something.
parallel—of, relating to, or designating lines, curves, planes, and/or or surfaces everywhere equidistant and/or an arrangement of components in an electrical circuit that splits an electrical current into two or more paths.
per—for each and/or by means of
perceptible—capable of being perceived by the human senses.
permanent—not temporary and/or not expected to change for an indefinite time.
perpendicular—of, relating to, or designating two or more straight coplanar lines or planes that intersect at approximately a right angle.
physical—tangible, real, and/or actual.
physically—existing, happening, occurring, acting, and/or operating in a manner that is tangible, real, and/or actual.
plurality—the state of being plural and/or more than one.
pole—one of two or more regions in a magnetized body at which the magnetic flux density is concentrated.
portion—a part, component, section, percentage, ratio, and/or quantity that is less than a larger whole. Can be visually, physically, and/or virtually distinguishable and/or non-distinguishable.
position—(n) a place and/or location, often relative to a reference point. (v) to place and/or locate.
pre-—a prefix that precedes an activity that has occurred beforehand and/or in advance.
precess—to move in a gyrating fashion and/or to move in or be subjected to precession.
predetermine—to determine, decide, and/or establish in advance.
prevent—to hinder, avert, and/or keep from occurring.
prior—before and/or preceding in time or order.
probability—a quantitative representation of a likelihood of an occurrence.
processor—a machine that utilizes hardware, firmware, and/or software and is physically adaptable to perform, via Boolean logic operating on a plurality of logic gates that form particular physical circuits, a specific task defined by a set of machine-implementable instructions. A processor can utilize mechanical, pneumatic, hydraulic, electrical, magnetic, optical, informational, chemical, and/or biological principles, mechanisms, adaptations, signals, inputs, and/or outputs to perform the task(s). In certain embodiments, a processor can act upon information by manipulating, analyzing, modifying, and/or converting it, transmitting the information for use by machine-implementable instructions and/or an information device, and/or routing the information to an output device. A processor can function as a central processing unit, local controller, remote controller, parallel controller, and/or distributed controller, etc. Unless stated otherwise, the processor can be a general-purpose device, such as a microcontroller and/or a microprocessor, such the Pentium family of microprocessor manufactured by the Intel Corporation of Santa Clara, Calif. In certain embodiments, the processor can be dedicated purpose device, such as an Application Specific Integrated Circuit (ASIC) or a Field Programmable Gate Array (FPGA) that has been designed to implement in its hardware and/or firmware at least a part of an embodiment disclosed herein. A processor can reside on and use the capabilities of a controller.
produce—to generate via a physical effort, manufacture, and/or make.
product—something produced by human and/or mechanical effort.
project—to calculate, estimate, or predict.
provide—to furnish, supply, give, convey, send, and/or make available.
pulse—a transient variation of a quantity (such as electric current or voltage) whose value is otherwise constant. Sometimes repeated with a regular period and/or according to some code.
Radio frequency—a frequency in the range within which radio waves may be transmitted, from about 3 kilohertz to about 300,000 megahertz.
range—a measure of an extent of a set of values and/or an amount and/or extent of variation.
ratio—a relationship between two quantities expressed as a quotient of one divided by the other.
receive—to gather, take, acquire, obtain, accept, get, and/or have bestowed upon.
recommend—to suggest, praise, commend, and/or endorse.
reduce—to make and/or become lesser and/or smaller.
relative—considered with reference to and/or in comparison to something else.
remove—to eliminate, remove, and/or delete, and/or to move from a place or position occupied.
render—to display, annunciate, speak, print, and/or otherwise make perceptible to a human, for example as data, commands, text, graphics, audio, video, animation, and/or hyperlinks, etc., such as via any visual, audio, and/or haptic mechanism, such as via a display, monitor, printer, electric paper, ocular implant, cochlear implant, speaker, etc.
render—to, e.g., physically, chemically, biologically, electronically, electrically, magnetically, optically, acoustically, fluidically, and/or mechanically, etc., transform information into a form perceptible to a human as, for example, data, commands, text, graphics, audio, video, animation, and/or hyperlinks, etc., such as via a visual, audio, and/or haptic, etc., means and/or depiction, such as via a display, monitor, electric paper, ocular implant, cochlear implant, speaker, vibrator, shaker, force-feedback device, stylus, joystick, steering wheel, glove, blower, heater, cooler, pin array, tactile touchscreen, etc.
repeat—to do again and/or perform again.
repeatedly—again and again; repetitively.
repetition—the act or an instance of repeating and/or a thing, word, action, etc., that is repeated.
request—to express a desire for and/or ask for.
responsive—reacting to an influence and/or impetus.
result—(n.) an outcome and/or consequence of a particular action, operation, and/or course; (v.) to cause an outcome and/or consequence of a particular action, operation, and/or course.
rich—having an abundant supply.
said—when used in a system or device claim, an article indicating a subsequent claim term that has been previously introduced.
second—an entity immediately following a first entity in an ordering.
select—to make a choice or selection from alternatives.
sensor—a device used to measure a physical quantity (e.g., temperature, pressure, capacitance, and/or loudness, etc.) and convert that physical quantity into a signal of some kind (e.g., voltage, current, power, etc.). A sensor can be any instrument such as, for example, any instrument measuring pressure, temperature, flow, mass, heat, light, sound, humidity, proximity, position, gap, count, velocity, vibration, voltage, current, capacitance, resistance, inductance, and/or electro-magnetic radiation, etc. Such instruments can comprise, for example, proximity switches, photo sensors, thermocouples, level indicating devices, speed sensors, electrical voltage indicators, electrical current indicators, on/off indicators, and/or flowmeters, etc.
sequential—ordered in time.
server—an information device and/or a process running thereon, that is adapted to be communicatively coupled to a network and that is adapted to provide at least one service for at least one client, i.e., for at least one other information device communicatively coupled to the network and/or for at least one process running on another information device communicatively coupled to the network. One example is a file server, which has a local drive and services requests from remote clients to read, write, and/or manage files on that drive. Another example is an e-mail server, which provides at least one program that accepts, temporarily stores, relays, and/or delivers e-mail messages. Still another example is a database server, which processes database queries. Yet another example is a device server, which provides networked and/or programmable: access to, and/or monitoring, management, and/or control of, shared physical resources and/or devices, such as information devices, printers, modems, scanners, projectors, displays, lights, cameras, security equipment, proximity readers, card readers, kiosks, POS/retail equipment, phone systems, residential equipment, HVAC equipment, medical equipment, laboratory equipment, industrial equipment, machine tools, pumps, fans, motor drives, scales, programmable logic controllers, sensors, data collectors, actuators, alarms, annunciators, and/or input/output devices, etc.
set—a related plurality of predetermined elements; and/or one or more distinct items and/or entities having a specific common property or properties.
signal—(v) to communicate; (n) one or more automatically detectable variations in a physical variable, such as a pneumatic, hydraulic, acoustic, fluidic, mechanical, electrical, magnetic, optical, chemical, and/or biological variable, such as power, energy, pressure, flowrate, viscosity, density, torque, impact, force, frequency, phase, voltage, current, resistance, magnetomotive force, magnetic field intensity, magnetic field flux, magnetic flux density, reluctance, permeability, index of refraction, optical wavelength, polarization, reflectance, transmittance, phase shift, concentration, and/or temperature, etc., that can encode information, such as machine-implementable instructions for activities and/or one or more letters, words, characters, symbols, signal flags, visual displays, and/or special sounds, etc., having prearranged meaning. Depending on the context, a signal and/or the information encoded therein can be synchronous, asynchronous, hard real-time, soft real-time, non-real time, continuously generated, continuously varying, analog, discretely generated, discretely varying, quantized, digital, broadcast, multicast, unicast, transmitted, conveyed, received, continuously measured, discretely measured, processed, encoded, encrypted, multiplexed, modulated, spread, de-spread, demodulated, detected, de-multiplexed, decrypted, and/or decoded, etc.
spacing—a separation.
special purpose computer—a computer and/or information device comprising a processor device having a plurality of logic gates, whereby at least a portion of those logic gates, via implementation of specific machine-implementable instructions by the processor, experience a change in at least one physical and measurable property, such as a voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc., thereby directly tying the specific machine-implementable instructions to the logic gate's specific configuration and property(ies). In the context of an electronic computer, each such change in the logic gates creates a specific electrical circuit, thereby directly tying the specific machine-implementable instructions to that specific electrical circuit.
special purpose processor—a processor device, having a plurality of logic gates, whereby at least a portion of those logic gates, via implementation of specific machine-implementable instructions by the processor, experience a change in at least one physical and measurable property, such as a voltage, current, charge, phase, pressure, weight, height, tension, level, gap, position, velocity, momentum, force, temperature, polarity, magnetic field, magnetic force, magnetic orientation, reflectivity, molecular linkage, molecular weight, etc., thereby directly tying the specific machine-implementable instructions to the logic gate's specific configuration and property(ies). In the context of an electronic computer, each such change in the logic gates creates a specific electrical circuit, thereby directly tying the specific machine-implementable instructions to that specific electrical circuit.
species—a class of individuals and/or objects grouped by virtue of their common attributes and assigned a common name; a division subordinate to a genus.
spin—a form of angular momentum carried by atomic nuclei.
spin echo—the refocusing of spin magnetisation by a pulse of resonant electromagnetic radiation.
spin-spin relaxation time—the time it takes for the magnetic resonance signal to irreversibly decay to 37% (1/c) of its initial value after its generation by tipping the longitudinal magnetization towards the magnetic transverse plane; and/or a mechanism by which the transverse component of a magnetization vector exponentially decays towards its equilibrium value in nuclear magnetic resonance, and which is characterized by the spin-spin relaxation time, which is a time constant characterizing the signal decay.
static—stationary and/or constant.
store—to place, hold, and/or retain data, typically in a memory.
substantially—to a considerable, large, and/or great, but not necessarily whole and/or entire, extent and/or degree.
such that—in a manner that results in.
sufficient—a degree and/or amount necessary to achieve a predetermined result.
sum—to add together and/or the result thereof.
support—to bear the weight of, especially from below.
surrounding—to encircle, enclose or confine on all sides, and/or extend on most and/or all sides of simultaneously.
switch—(v) to: form, open, and/or close one or more circuits; form, complete, and/or break an electrical and/or informational path; select a path and/or circuit from a plurality of available paths and/or circuits; and/or establish a connection between disparate transmission path segments in a network (or between networks); (n) a physical device, such as a mechanical, electrical, and/or electronic device, that is adapted to switch.
system—a collection of mechanisms, devices, machines, articles of manufacture, processes, data, and/or instructions, the collection designed to perform one or more specific functions.
terminal—of, at, relating to, or forming a limit, boundary, extremity, or end.
that—a pronoun used to indicate a thing as indicated, mentioned before, present, and/or well known.
through—across, among, between, and/or in one side and out the opposite and/or another side of.
time—a measurement of a point in a nonspatial continuum in which events occur in apparently irreversible succession from the past through the present to the future.
time constant—the time required for a variable to rise or fall exponentially through approximately 63 percent of its amplitude.
time-dependent—varying over time.
time-varying—changing with respect to time and/or not temporally static.
to—a preposition adapted for use for expressing purpose.
together—into a unified arrangement.
train—a sequence and/or orderly succession of related events.
transform—to change in measurable: form, appearance, nature, and/or character.
transmit—to send as a signal, provide, furnish, and/or supply.
transverse—situated or lying across; crosswise; at a right angle to a long axis of a body.
treatment—an act, manner, or method of handling and/or dealing with someone and/or something.
upon—immediately or very soon after; and/or on the occasion of
use—to put into service.
user interface—any device for rendering information to a user and/or requesting information from the user. A user interface includes at least one of textual, graphical, audio, video, animation, and/or haptic elements. A textual element can be provided, for example, by a printer, monitor, display, projector, etc. A graphical element can be provided, for example, via a monitor, display, projector, and/or visual indication device, such as a light, flag, beacon, etc. An audio element can be provided, for example, via a speaker, microphone, and/or other sound generating and/or receiving device. A video element or animation element can be provided, for example, via a monitor, display, projector, and/or other visual device. A haptic element can be provided, for example, via a very low frequency speaker, vibrator, tactile stimulator, tactile pad, simulator, keyboard, keypad, mouse, trackball, joystick, gamepad, wheel, touchpad, touch panel, pointing device, and/or other haptic device, etc. A user interface can include one or more textual elements such as, for example, one or more letters, number, symbols, etc. A user interface can include one or more graphical elements such as, for example, an image, photograph, drawing, icon, window, title bar, panel, sheet, tab, drawer, matrix, table, form, calendar, outline view, frame, dialog box, static text, text box, list, pick list, pop-up list, pull-down list, menu, tool bar, dock, check box, radio button, hyperlink, browser, button, control, palette, preview panel, color wheel, dial, slider, scroll bar, cursor, status bar, stepper, and/or progress indicator, etc. A textual and/or graphical element can be used for selecting, programming, adjusting, changing, specifying, etc. an appearance, background color, background style, border style, border thickness, foreground color, font, font style, font size, alignment, line spacing, indent, maximum data length, validation, query, cursor type, pointer type, autosizing, position, and/or dimension, etc. A user interface can include one or more audio elements such as, for example, a volume control, pitch control, speed control, voice selector, and/or one or more elements for controlling audio play, speed, pause, fast forward, reverse, etc. A user interface can include one or more video elements such as, for example, elements controlling video play, speed, pause, fast forward, reverse, zoom-in, zoom-out, rotate, and/or tilt, etc. A user interface can include one or more animation elements such as, for example, elements controlling animation play, pause, fast forward, reverse, zoom-in, zoom-out, rotate, tilt, color, intensity, speed, frequency, appearance, etc. A user interface can include one or more haptic elements such as, for example, elements utilizing tactile stimulus, force, pressure, vibration, motion, displacement, temperature, etc.
value—a measured, provided, assigned, determined, and/or calculated quantity or quality for a variable and/or parameter.
variation—the state, fact, act, process, and/or result of varying.
varies—changes over time.
via—by way of, with, and/or utilizing.
wait—pause.
weight—a force with which a body is attracted to Earth or another celestial body, equal to the product of the object's mass and the acceleration of gravity; and/or a factor and/or value assigned to a number in a computation, such as in determining an average, to make the number's effect on the computation reflect its importance, significance, preference, impact, etc.
when—at a time and/or during the time at which.
wherein—in regard to which; and; and/or in addition to.
which—a pronoun adapted to be used in clauses to represent a specified antecedent.
while—for as long as, during the time that, and/or at the same time that.
with—accompanied by.
with respect to—about, regarding, relative to, and/or in relation to.
within—inside the limits of.
zone—a region and/or volume having at least one predetermined boundary.

### Note

Various substantially and specifically practical and useful exemplary embodiments of the claimed subject matter are described herein, textually and/or graphically, including the best mode, if any, known to the inventor(s), for implementing the claimed subject matter by persons having ordinary skill in the art. Any of numerous possible variations (e.g., modifications, augmentations, embellishments, refinements, and/or enhancements, etc.), details (e.g., species, aspects, nuances, and/or elaborations, etc.), and/or equivalents (e.g., substitutions, replacements, combinations, and/or alternatives, etc.) of one or more embodiments described herein might become apparent upon reading this document to a person having ordinary skill in the art, relying upon his/her expertise and/or knowledge of the entirety of the art and without exercising undue experimentation. The inventor(s) expects skilled artisans to implement such variations, details, and/or equivalents as appropriate, and the inventor(s) therefore intends for the claimed subject matter to be practiced other than as specifically described herein. Accordingly, as permitted by law, the claimed subject matter includes and covers all variations, details, and equivalents of that claimed subject matter. Moreover, as permitted by law, every combination of the herein described characteristics, functions, activities, substances, and/or structural elements, and all possible variations, details, and equivalents thereof, is encompassed by the claimed subject matter unless otherwise clearly indicated herein, clearly and specifically disclaimed, or otherwise clearly contradicted by context.

The use of any and all examples, or exemplary language (e.g., “such as”) provided herein, is intended merely to better illuminate one or more embodiments and does not pose a limitation on the scope of any claimed subject matter unless otherwise stated. No language herein should be construed as indicating any non-claimed subject matter as essential to the practice of the claimed subject matter.

Thus, regardless of the content of any portion (e.g., title, field, background, summary, description, abstract, drawing figure, etc.) of this document, unless clearly specified to the contrary, such as via explicit definition, assertion, or argument, or clearly contradicted by context, with respect to any claim, whether of this document and/or any claim of any document claiming priority hereto, and whether originally presented or otherwise:
there is no requirement for the inclusion of any particular described characteristic, function, activity, substance, or structural element, for any particular sequence of activities, for any particular combination of substances, or for any particular interrelationship of elements;
no described characteristic, function, activity, substance, or structural element is “essential”;
any two or more described substances can be mixed, combined, reacted, separated, and/or segregated;
any described characteristics, functions, activities, substances, and/or structural elements can be integrated, segregated, and/or duplicated;
any described activity can be performed manually, semi-automatically, and/or automatically;
any described activity can be repeated, any activity can be performed by multiple entities, and/or any activity can be performed in multiple jurisdictions; and
any described characteristic, function, activity, substance, and/or structural element can be specifically excluded, the sequence of activities can vary, and/or the interrelationship of structural elements can vary.

The use of the terms “a”, “an”, “said”, “the”, and/or similar referents in the context of describing various embodiments (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted.

When any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate subrange defined by such separate values is incorporated into the specification as if it were individually recited herein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any phrase (i.e., one or more words) appearing in a claim is followed by a drawing element number, that drawing element number is exemplary and non-limiting on claim scope.

No claim of this document is intended to invoke paragraph six of 35 USC 112 unless the precise phrase “means for” is followed by a gerund.

Any information in any material (e.g., a United States patent, United States patent application, book, article, etc.) that has been incorporated by reference herein, is incorporated by reference herein in its entirety to its fullest enabling extent permitted by law yet only to the extent that no conflict exists between such information and the other definitions, statements, and/or drawings set forth herein. In the event of such conflict, including a conflict that would render invalid any claim herein or seeking priority hereto, then any such conflicting information in such material is specifically not incorporated by reference herein. Any specific information in any portion of any material that has been incorporated by reference herein that identifies, criticizes, or compares to any prior art is not incorporated by reference herein.

Within this document, and during prosecution of any patent application related hereto, any reference to any claimed subject matter is intended to reference the precise language of the then-pending claimed subject matter at that particular point in time only.

Accordingly, every portion (e.g., title, field, background, summary, description, abstract, drawing figure, etc.) of this document, other than the claims themselves and any provided definitions of the phrases used therein, is to be regarded as illustrative in nature, and not as restrictive. The scope of subject matter protected by any claim of any patent that issues based on this document is defined and limited only by the precise language of that claim (and all legal equivalents thereof) and any provided definition of any phrase used in that claim, as informed by the context of this document.

What is claimed is:

1. A method comprising:
via one or more predetermined processors communicatively coupled to sensor coil of a nuclear magnetic resonance instrument:
determining a radio frequency that substantially matches a hydrogen nuclei Larmor frequency for a capillary-rich portion of a digit of a mammal, the capillary-rich portion containing a large amount of blood relative to a bone portion of the digit, the hydrogen nuclei Larmor frequency corresponding to a static magnetic field induced by one or more permanent magnets to cross an air gap between an opposing pair of pole faces that have a transverse spacing sufficient to receive a cup that is configured to receive the digit, a magnitude of the hydrogen nuclei Larmor frequency dependent on a position of the portion of the digit between the pair of pole faces, the radio frequency a measure of time-dependent variation in a longitudinal magnetic field induced by a time-varying current in the sensor coil, the sensor coil substantially surrounding the cup and defining a coil axis oriented substantially parallel to a longitudinal axis of the cup;
while the longitudinal magnetic field is applied to the digit, acquiring an amplitude and a spin-spin relaxation time of each of a train of spin echoes created by applying a plurality of CPMG pulses to the digit via the sensor coil, a count of the spin echoes in the train of spin echoes corresponding to a decay of the spin echo amplitudes to a predetermined value;
based on the amplitudes of the spin echoes, determining a distribution of spin-spin relaxation time constants of a plurality of components in the blood; and
for each of the one or more predetermined components, based on the distribution of spin-spin relaxation time constants, determining a relative concentration of the predetermined component in the blood.

2. The method of claim 1, further comprising:
repeating said acquiring for a predetermined number of repetitions.

3. The method of claim 1, further comprising:
repeating said acquiring for a predetermined number of repetitions, each repetition delayed by a wait time that is greater than a spin lattice relaxation time of one or more predetermined components of the plurality of components.

4. The method of claim 1, further comprising:
repeating said acquiring for a predetermined number of repetitions, each repetition delayed by a wait time that is less than a spin lattice relaxation time of one or more predetermined components of the plurality of components.

5. The method of claim 1, further comprising:
repeating said acquiring for a predetermined number (N) of repetitions such that a plurality of echo trains is acquired, each echo train comprising a plurality echoes, each echo from each echo train having a corresponding sequential position in that echo train; and
for the plurality of echo trains, for each sequential position, summing an amplitude of the corresponding echoes, such that all first echoes are summed together, all second echoes are summed together, and all N echoes are summed together.

6. The method of claim 1, further comprising:
repeating said acquiring for a predetermined number of repetitions; and
summing similarly timed echoes across the predetermined number of repetitions.

7. The method of claim 1, further comprising:
rendering the relative concentration of the one or more predetermined component in the blood.

8. The method of claim 1, wherein:
at least one of the one or more processors is communicatively coupled to the sensor coil via a network.

9. A method comprising:
via one or more predetermined processors communicatively coupled to sensor coil of a nuclear magnetic resonance instrument:
for each of one or more predetermined components of a plurality of components in blood of a digit of a mammal, based on a distribution of spin-spin relaxation time constants for hydrogen nuclei of the predetermined component, determining a relative concentration of the predetermined component in the blood, the distribution of spin-spin relaxation time constants determined based on amplitudes of a train of spin echoes created by a plurality of CPMG pulses applied to the digit by a sensor coil while a longitudinal magnetic field is applied to the digit, a count of the spin echoes in the train of spin echoes corresponding to decay of the spin echoes to a predetermined value, the sensor coil substantially surrounding a cup and defining a coil axis oriented substantially parallel to a longitudinal axis of the cup, the cup configured to receive the digit, the cup located within a transverse spacing between an opposing pair of pole faces of one or more permanent magnets, the transverse spacing defining an air gap across which the one or more permanent magnets are configured to produce a static magnetic field, the static magnetic field configured to induce hydrogen nuclei of the digit to precess at a corresponding Larmor frequency, the Larmor frequency of each hydrogen nuclei having a magnitude that is dependent on a position of a portion of the digit between the pair of pole faces, a time-dependent variation in the longitudinal magnetic field applied by a time-varying current in the sensor coil having a frequency substantially matching the Larmor frequency for a capillary-rich portion of the digit, the capillary-rich portion containing a large amount of blood relative to a bone portion of the digit.

10. A method comprising:
via one or more predetermined processors communicatively coupled to sensor coil of a nuclear magnetic resonance instrument:
determining a radio frequency that substantially matches a hydrogen nuclei Larmor frequency for a capillary-rich portion of a digit of a mammal, the capillary-rich portion containing a large amount of blood relative to a bone portion of the digit, the hydrogen nuclei Larmor frequency corresponding to a static magnetic field induced one or more permanent magnets to cross an air gap between an opposing pair of pole faces that have a transverse spacing sufficient to receive a cup that is configured to receive the digit, a magnitude of the hydrogen nuclei Larmor frequency dependent on a position of the portion of the digit between the pair of pole faces, the radio frequency a measure of time-dependent variation in a longitudinal magnetic field induced by a time-varying current in the sensor coil, the sensor coil substantially surrounding the cup and defining a coil axis oriented substantially parallel to a longitudinal axis of the cup.

11. A device, comprising:
a cup configured to receive at least a terminal portion of a digit of a mammal, the cup defining a cup longitudinal axis;
one or more permanent magnets configured to induce a static magnetic field to cross an air gap located between an opposing pair of pole faces that have a transverse spacing sufficient to receive the cup;
a sensor coil substantially surrounding the cup, defining a coil axis oriented substantially parallel to a longitudinal axis of the cup, and configured to produce a longitudinal magnetic field that varies with respect to time responsive to application of a time-varying current to the sensor coil;
wherein:
the static magnetic field is configured to induce hydrogen nuclei of the digit to precess at a corresponding Larmor frequency;
the Larmor frequency of each hydrogen nuclei has a magnitude that is dependent on a position of a portion of the digit between the pair of pole faces;
the time-dependent variation in the longitudinal magnetic field has a frequency substantially matching a Larmor frequency for a capillary-rich portion of the digit, the capillary-rich portion containing a large amount of blood relative to a bone portion of the digit.

12. A device, comprising:
a cup configured to receive at least a terminal portion of a digit of a mammal, the cup defining a cup longitudinal axis;
one or more permanent magnets configured to induce a static magnetic field to cross an air Thanks Kelly.
gap located between an opposing pair of pole faces that have a transverse spacing sufficient to receive the cup;
a sensor coil substantially surrounding the cup, defining a coil axis oriented substantially parallel to a longitudinal axis of the cup, and configured to produce a longitudinal magnetic field that varies with respect to time responsive to application of a time-varying current to the sensor coil;
wherein:
the sensor coil is configured to acquire a train of spin echoes created by a plurality of CPMG pulses applied to the digit by a sensor coil while the longitudinal magnetic field is applied to the digit, the train of spin echoes defining amplitudes and corresponding spin-spin relaxation times, the amplitudes and spin-spin relaxation times corresponding to a distribution of spin-spin relaxation time constants for hydrogen nuclei of a predetermined component of a plurality of components of blood of the mammal, the distribution corresponding to a relative concentration of the predetermined component in the blood.
